(19) United States
(12) Patent Application Publication
(10) Pub. No.: US 2002/0092037 A1
(43) Pub. Date: Jul. 11, 2002

(54) Use of membrane supports in plant tissue culture processes

(76) Inventors: Marie Bernice Connett-Porceddu, Summerville, SC (US); James Douglas Allnutt, Ladson, SC (US); Heather Jeanne Gladfelter, North Charleston, SC (US); Jon Eric Gulledge, Goose Creek, SC (US); Ryan Robert McCormack, Ithaca, NY (US)

Correspondence Address: Westvaco Corporation, 5255 Virginia Avenue, Post Office Box 118005, Charleston, SC, 29423-8005, US

(21) Appl. No.: 09/973,369

(22) Filed: Oct. 9, 2001

Related U.S. Application Data
(60) Provisional application No. 60/239,143, filed on Oct. 10, 2000.

(51) Int. Cl. C12N 15/82; A01H 4/00; A01H 5/04

(52) U.S. Cl. 800/278; 435/420; 435/422; 435/431; 800/319

(57) ABSTRACT

This invention relates to the use of highly liquid-permeable membrane supports, made from low-absorption fibers such as polyester and other non-cellulosic fibers with similar characteristics, in plant tissue culture processes. In particular, the invention relates to the use of such supports over gelled media or over a bilayer liquid/gelled system to improve the growth and embryogenic or organogenic development of plant cell cultures, the selection of transformant plant cells, and the decontamination of plant cell cultures of fungal and/or bacterial contaminants.

This application claims the benefit of U.S. Provisional Application No. 60/239,143, filed on Oct. 10, 2000.

### FIELD OF INVENTION

This invention relates to the use of highly liquid-permeable membrane supports, made from low-absorption fibers such as polyester and other non-cellulosic fibers with similar characteristics, in plant tissue culture processes. In particular, the invention relates to the use of such supports over gelled media or over a bilayer liquid/gelled system to improve the growth and development of plant cell cultures, the selection of transformant plant cells, and the eradication of bacterial contaminants in plant cell cultures.

This invention relates to the use of highly liquid-permeable membrane supports, made from low-absorption fibers such as polyester and other non-cellulosic fibers with similar characteristics, in plant tissue culture processes. In particular, the invention relates to the use of such supports over gelled media or over a bilayer liquid/gelled system to improve the growth and embryogenic or organogenic development of plant cell cultures, the selection of transformant plant cells, and the decontamination of plant cell cultures of fungal and/or bacterial contaminants.

### BACKGROUND OF THE INVENTION

It is a common practice in tissue culture processes to transfer cultured cells from one medium vessel (e.g., a suspension flask containing liquid medium or a plate containing gelled medium) to another in order to replenish nutrients and plant growth regulators that have been depleted during the period of culture. It is also a standard practice to transfer cultured cells from one medium vessel to another in order to add or remove a component in the medium. Examples of such components include substrates such as antibiotics or herbicides used for selection, eradicants used for decontamination, and plant growth regulator/substrate combinations that modulate a developmental switch (such as rooting, shoot proliferation, embryo maturation and the like).

Cells maintained in a liquid suspension culture medium can be difficult to transfer without carryover from the previous medium. The method usually employed for such transfers is to pour the cell suspensions over a solid support consisting of either filter paper or some type of membrane (usually nylon) wherein the solid support may be placed on a funnel, Buchner funnel, or similar apparatus for removal by gravity, vacuum, or capillary action of the liquid medium from the cells left on the solid support. Subsequently, the cells are removed from the solid support into the fresh medium. However, a problem exists with the use of filter paper as the solid support in this method due to the fact that many of the cells tend to adhere to the filter paper, thereby making it difficult to remove the cells into fresh medium. It is similarly difficult to remove cells completely where nylon membrane solid supports are employed, as the cells often tend to adhere to the nylon. These adherence problems can necessitate a significant amount of agitation and scraping to remove the cells from the solid supports—actions that potentially damage many of the cells being transferred.

Friable plant cell clumps such as embryogenic tissue or callus maintained on the surface of a gelled medium can also be quite cumbersome to transfer, as plant tissues tend to become partially embedded in the gelled culture medium. This problem can be exacerbated as plant cells acidify the surrounding medium, causing partial depolymerization of some gelling agents and resulting in further sinking into the medium. Incomplete transfer from the medium surface often results in the loss of many of the plant cells. Some cell technicians have attempted to address this problem via the process of maintaining cells on a filter paper solid support or other cellulosic-based solid supports (such as MILLIPORE GS® and the like) which are laid over the surface of the gelled medium. The use of nylon mesh membranes for the specific purpose of ease of transfer has also been practiced (U.S. Pat. Nos. 5,141,866, 5,238,835, 5,445,961 and 5,506,136), as has the use of nylon mesh for supporting thin alginate layers in which protoplast regeneration can be monitored (Korlach and Zoglauer 1995). However, cells so maintained tend to exhibit the same adherence problems as those found with the use of solid supports for liquid suspension cultures and are similarly difficult to remove from the supports later.

Additionally, many types of plant cell cultures grown directly on gelled media exhibit increased necrosis in the cell layers that are directly on or partially submerged in the gelled media. This has been attributed to anaerobicity or undesirable water potential in these locations. These necrotic cell layers can slow or prevent penetration of the upper layers of tissue by plant growth regulators and other compounds present in the underlying medium (such as antibiotics or herbicides used in the selection of transformed cells after a genetic engineering process). Furthermore, shoot or embryo regeneration from callus or callus-like masses growing on or partially submerged in the gelled media may be compromised; for example, some callus or shoot material can form fused clusters with poor internal gas exchange and poor separability (e.g. Teng 1997).

Effects on growth or development of the cells when using nylon membranes for ease of transfer, relative to not using nylon membranes, have not been taught in the patents and articles mentioned above. We have found that while cells may be separated from the gelled media via the use of a cellulosic-based or nylon (U.S. Pat. Nos. 5,141,866, 5,238,835, 5,445,961 and 5,506,136) membrane solid support, for many types of plant cell cultures the same unfavorable necrosis-promoting conditions often exist at the surface of the support. Furthermore, penetration may also be reduced by the use of supports that are semi-impermeable or non-conductive with respect to the nutrients, plant growth regulators and other compounds present in the underlying medium. For example, we have found that vital staining dyes do not flow rapidly from the underlying medium through nylon membrane solid supports (possibly due to swelling of the nylon fibers when wetted, with a possible additional contribution by adsorption by the nylon fibers due to slight reactivity under the acidic conditions often prevailing in plant cell cultures). In contrast, the vital staining dyes were able to pass rapidly through membranes made of non-swelling, non-absorbent artificial fibers such as polyester, from underlying gelled media or from a thin layer of liquid spread or poured over gelled media, into cells cultured on the membranes. The use of vital staining dyes allows visualization of the probable pathway and/or barriers likely to be experienced by other relatively large molecules (which could comprise antibiotics or other selection agents or eradicants, multimeric sugars and other polymers, plant growth regulators, and the like).

Where penetration of molecules such as plant growth regulators, carbon sources, selection agents or eradicants is hampered by necrosis, adsorption, or necrosis-promoting conditions at the surface of the gelled medium (or the nylon or cellulosic-based support), higher doses of these compounds may be needed, and it is less likely that homogeneous doses will reach all parts of the tissue. In the case of selection after transformation, there can be a significant frequency of cells that tend to “escape” selection agents that are administered by incorporation in the gelled medium, thereby adding to the difficulty of screening for transformation events and/or increasing the likelihood that chimaerae will arise. In the case of eradicants administered by incorporation in the gelled medium on which plant tissue is cultured to clear microbial contamination, there can be a significant frequency of microbial “escape” as well, leading to the reappearance of contamination, and larger doses must often be the resort.

Often additional problems exist which are associated with the penetration of the cell tissue by plant growth regulators and other compounds present in the underlying medium. For example, some media components exhibit less efficient rates of diffusion into plant cells across a gelled matrix when compared with the rates of diffusion of the components in aqueous solution. One method of dealing with this limitation of gelled media has been to grow plant material in liquid culture, or even in a double-phase culture system using liquid medium laid over gelled medium. A number of publications (e.g., Aitken-Christie and Jones 1987, Molnar 1987, Viseur 1987) teach the method for differentiated shoot cultures. Because such a method increases the availability of water and avoids drying of the explants, it has also been useful in culture of delicate structures such as protoplasts (U.S. Pat. No. 5,166,068, He et al. 1996), embryo sacs (Mol et al. 1995) and isolated proembryos (Wang et al. 1996). However, many types of differentiated and non-differentiated plant cells, as they grow and develop, show detrimental vitrification or hyperhydricity when grown in such liquid culture or partial liquid culture systems (John 1986, Romano et al. 1992). A solution to the vitrification problem for some plant culture systems has been the use of “wicks” or filter paper “bridges” conveying liquid into the tissue from liquid reservoirs (Pillai and Hildebrandt 1969, Tokomasu and Kato 1978) or “rafts” or similar types of supports floated over volumes of liquid media (e.g. Teng 1997, U.S. Pat. No. 5,445,961, U.S. Pat. No. 6,200,809). However, unless some sort of support apparatus is used (e.g. U.S. Pat. Nos. 4,531,324 and 5,141,866) the weight gain of the growing plant material can cause the bridge or raft to sink below the level of the liquid, so that anaerobicity and vitrification resume unless material is regularly removed from the raft (U.S. Pat. No. 4,531,324 and OSMOTEK® product literature), with the same difficulties of damage during removal as mentioned above. A discussion of this problem is included in the text of U.S. Pat. No. 5,141,866, which claims the use of a sterile nylon mesh “screening element” supported over liquid culture medium. Tissue homogenates from which re-differentiation was desired were found to be difficult to remove with a spatula, a process which also frequently introduced contamination. The patent claimed an improvement to the process, which still required extensive washing to remove tissue from the nylon screens in flasks. Working with rafts or bridges (with or without support posts) carrying tissue floating on volumes of liquid can also be cumbersome and contamination-prone. The use of thick polyester pads, also known as “batting” or “sponges” immersed in liquid media has also been taught for use specifically as an aerated support in liquid media for development of embryos of certain species (U.S. Pat. No. 5,236,841). However, no growth advantage is taught or claimed in the patent for this method—indeed, because of low numbers of embryos produced it is not preferred over the use of filter paper on gelled medium. Removal of developed embryos that have become entwined in the thick fibrous pads can also be cumbersome. Thus, it remains a more common practice to culture cells on gelled medium despite the concurrent lack of penetration of some of the cells.

Contaminating bacteria or fungi of any kind are hazards to sterile plant cell cultures because the nutrient medium supporting the growth of the plant cells may also allow the continued growth of the microbial contaminants, which may overgrow the plant material and compete with it for nutrients. Eradication of microbial contaminants once introduced into plant cell cultures has been the subject of much endeavor, and too often valuable cultures are discarded because of a failure to eradicate a contaminant. Traditional methods of eradication (or decontamination) involve washing and multiple transfers of the plant cells into uncontaminated media (which often entails the adherence difficulties described above), and/or the incorporation into the medium of eradicant(s) such as fungistatic or fungicidal compounds or antibiotics such as ticarcillin, carbenicillin, or a cephalosporin. Agrobacterium contamination has been reported as recurring sometimes after long periods of time, so with many species, eradicants are incorporated in all culture media used after the initial infection, including selection media, proliferation media, media to induce the formation of organs or the development of somatic embryos, media to elongate or mature organs or embryos that are formed, and regeneration media. Unfortunately, it is often difficult to incorporate these antimicrobial agents or eradicants into gelled media due to reactivity or inactivation of the eradicant at the temperature of gel polymerization. For example, with pine embryogenic cells, incorporation of eradicants into the embryo development and maturation media has been difficult due to the high temperature of polymerization of the media resulting from the incorporation of a high level of polyethylene glycol. Moreover, the continuous incorporation of these eradicants in culture media is relatively expensive.

Transformation via Agrobacterium spp. is an effective method for introducing new genes into certain plants. However, following transformation (which occurs during incubation of the plant material with Agrobacterium) the bacterial cells are contaminants to the resulting cell cultures, and thus Agrobacterium transformation is normally followed by one or more of the eradication methods listed above, with all their inherent difficulties and constraints.

Not only antimicrobial agents, but plant growth regulators and other large molecules important to the growth or development of plant materials in culture may be difficult to incorporate into gelled media for a variety of reasons, such as: reactivity or inactivation of the component at the temperature of gel polymerization, desire to set up a gradient in concentration or osmotic pressure relative to a particular component, or undesirability of incorporating particular components (e.g., high molecular weight sugars, polymers such as polyethylene glycol, and the like) into gelled media because such components can affect characteristics of the medium such as polymerization temperature or water tension in the gel. Methods for applying certain plant growth regulators and sugars to plant cultures in a thin layer of liquid media spread or poured over gelled media in a “bi-phasic” system have been described (Fisher and Neuhaus 1995, Piqueras et al. 1998), but they are constrained by the same limitations as the “biphasic” systems mentioned above (i.e., hyperhydricity and difficulty of manipulation).

Therefore, an object of this invention is to solve these major problems by disclosing an improved method for growing plant cell cultures on gelled medium.

Another object of this invention is to disclose an improved method for regenerating plants from plant cell cultures on gelled medium.

A further object of the present invention is to provide an improved method for growing plant cell cultures on “double layer” or “biphasic” culture systems comprised of two gelled phases or a gelled phase overlaid with liquid medium, and/or regenerating plants from plant cell cultures on “double layer” or “biphasic” culture systems (comprised of two gelled phases or a gelled phase overlaid with liquid medium).

Yet another object of the present invention is to provide an improved method for selecting genetically transformed plant cells.

A further object of the present invention is to provide improved methods for eradicating microbial contaminants from plant cell cultures.

Yet another object of the present invention is to provide an improved method for producing transgenic plants.

The publications and other materials used herein to illuminate the background of the invention or provide additional details respecting the practice, are incorporated by reference, and for convenience are respectively grouped in the appended Bibliography.

### SUMMARY OF THE INVENTION

The above objects are achieved by the use of certain fiber supports with various plant cell cultures. More specifically, the growth of plant cell callus on gelled medium can be improved via the method of placing a non-cellulosic based support made of acid-resistant fibers which do not swell appreciably (“non-swelling”) between the culture and the supporting gel medium. Likewise, the growth and development of plant cell embryos on gelled medium can be improved via the method of placing a support made of non-swelling fibers between the culture and the supporting gel medium. Furthermore, regeneration of shoots or roots from callus can be improved via the method of placing a thin non-cellulosic based support made of non-swelling acid-resistant fibers which are easily penetrable by plant growth factors between the culture and the supporting gel medium.

The transfer of plant material between gelled and liquid media can be improved by the use of a non-swelling fiber support such as a polyester, polypropylene, or fluoropolymer membrane to which the plant material does not cling as readily as it does to cellulosic-based supports or fabric supports made of fibers such as nylon that swell appreciably when in contact with liquid. The transfer of plant material between liquid and gelled media, or between different gelled media, facilitated by the use of nylon or cellulosic-based supports as previously taught in the literature, can be improved using a support made of non-swelling fibers, such as a polyester or fluoropolymer membrane, through which media components may penetrate more readily.

The growth and development of plant cell cultures on “double layer” or “biphasic” culture systems comprised of two gelled phases or a gelled phase overlaid with liquid medium can be improved via the method of employing a thin non-cellulosic based support made of non-swelling, acid-resistant fibers which are easily penetrable by plant growth factors and other large molecules between the liquid and the plant tissue. The use of a thin non-cellulosic based support made of non-swelling, acid-resistant fibers over “double layer” or “biphasic” culture systems comprised of two gelled phases or a gelled phase overlaid with liquid medium, facilitates rapid and complete culture transfers. Regeneration of shoots or roots from plant cell cultures on “double layer” or “biphasic” culture systems comprised of two gelled phases or a gelled phase overlaid with liquid medium can be improved via the method of placing a thin non-cellulosic based fiber support which is easily penetrable by plant growth factors and other large molecules between the liquid and the plant tissue.

The efficiency of supplying to plant cells any large molecule such as any selection agent, antimicrobial agent, plant growth regulator, and/or polymer can be improved via the method of incorporating these molecules into liquid media for overlay on gelled media to create a “double layer” or “biphasic” culture system on which is laid a support made of non-swelling fibers between the liquid and the plant tissue.

The growth or selection of genetically transformed plant cells and the penetration of selective agents resulting in death of non-transformed cells can be improved via the method of placing a support made of non-swelling fibers between the cells and the medium containing the selection agent.

The eradication of microbial contaminants in plant cell cultures can be improved via the method of placing a support made of non-swelling fibers through which eradicants may penetrate more readily between the cell cultures and the medium containing the eradicant. Likewise, the washing of plant material in liquid media and replacement of the cells on gelled media can be improved by the use of a non-swelling fiber support to which the plant material does not cling as readily as it does to cellulosic-based supports or fabric supports made of fibers such as nylon (which swell appreciably when in contact with liquid).

### DESCRIPTION OF THE PREFERRED EMBODIMENT

We have found that certain constituents of tissue culture media (such as nutrients, plant growth regulators, polymers and the like) readily pass through certain non-cellulosic based support membranes. Thus, as an alternative to maintaining, developing, maturing, or regenerating plant cultures on the surface of the gelled media (wherein the cells tend to become partially embedded) or on filter papers or cellulosic pads (which can adsorb components of the media), cultures may be grown on non-cellulosic based support membranes placed over gelled nutrient media. The effect is particularly pronounced when the support membranes are made of a fiber or material that does not swell appreciably as a result of taking up and retaining liquid from the medium.

Specifically, experiments have shown that the growth rate and regenerability of a variety of plant cell cultures, including tobacco, cottonwood, sweetgum, Eucalyptus, pine, and Arabidopsis, these cultures being proliferative, embryogenic, or organogenic, and derived from herbaceous, woody angiosperm, or gymnospern plants, on certain non-cellulosic based support membranes over gelled media to be either equal to, or superior to, the growth rate of cultures maintained directly on the gelled media or on nylon membranes. A growth and regenerability advantage associated with the use of such support membranes was conferred in many cases due to a decreased formation of necrotic regions (which commonly appeared in cultures maintained on gelled or biphasic media without such support membranes, in the wet and anaerobic spaces directly adjacent to the surface of the medium). Moreover, the use of such support membranes permits the tissue culture callus to be spread thinly over the surface of membranes, thereby preventing clumps from becoming partially embedded in the media (and consequently becoming anaerobic), while still enabling the media components to reach the cultures over the entire surface via capillary action.

Non-cellulosic based support membranes that are suitable for use in the present invention include liquid-permeable support membranes prepared from materials made of low-absorption fibers. It is preferred that the liquid-permeable support membranes be prepared from material selected from the group consisting of polyesters, polypropylenes, fluoropolymers, and combinations thereof. It is further preferred that the materials be woven into a fabric, rather than pressed, to form the liquid-permeable support membranes. These materials exhibit both a decreased retention of liquid media within the fibers and a resistance to the mildly acidic conditions that often prevail in plant tissue cultures. A range of mesh sizes has been tested; and it is believe that pore sizes ranging from about 0.2 microns (to allow permeability to liquid medium and complex organic molecules) up to about half the size of the cells being cultured (to avert loss of the cultured cells through the mesh) can be used. As noted above, it is often quite difficult to remove cells completely where nylon membrane solid supports are employed, as the cells often tend to adhere among the swollen nylon fibers (which also result in a greatly decreased effective mesh size in the wetted membranes). The decreased effective mesh size can result in poor penetration of large molecules, and the adherence of cell culture material among the swollen fibers can necessitate a significant amount of agitation and scraping to remove the cells from the solid supports—actions which potentially damage many of the cells that are being transferred. It can also be difficult to remove tissue completely from filter papers or thick fibrous pads such as polyester, nylon or cellulosic “batting”, “felting” or “sponges” because the tissue becomes entwined in the surface fibers. Thus, the use of smooth polyester, polypropylene, or liquid-permeable fluoropolymer support membranes is preferred over nylon or over thick fibrous or felted pads because the use averts both the cell adherence problem and the lack of penetration by macromolecules such as plant growth regulators, polymers, selection agents, eradicants, and the like.

To summarize, an improved method for growing, culturing, maintaining, and/or regenerating a plant cell culture or plant callus comprises the culturing of cells or callus on a support membrane placed in contact with (“over”) the surface of gelled or biphasic support medium. This improved method can also be employed with genetically transformed cells and calli.

An improved method for plant cell tissue culture comprises culturing plant cells on a support membrane placed over a gel medium, wherein the improvement comprises the use of a liquid-permeable support membrane prepared from low-absorption fibers. Where desired, the plant cells can be plated onto the support membrane prior to culturing. It is preferred that the liquid-permeable support membrane be prepared from material selected from the group consisting of polyesters, polypropylenes, liquid-permeable fluoropolymers, and combinations thereof.

Where desired, the support membrane can be placed over a layer containing one or more culture components, said layer being positioned on said gel medium. It is preferred that the layer be a thin film of liquid medium or a filter paper with a liquid medium absorbed therein.

A second improvement is an improved method for growing plant cell tissues which comprises culturing plant cells on a support membrane placed over a gel medium, wherein the improvement comprises the use of a liquid-permeable support membrane prepared from low-absorption fibers that permits the diffusion of components from the medium to the plant cells to promote the growth of the plant cells above the medium without allowing the plant cells to become embedded in the medium. Where desired, the plant cells can be plated onto the support membrane prior to culturing. It is preferred that the liquid-permeable support membrane be prepared from material selected from the group consisting of polyesters, polypropylenes, liquid-permeable fluoropolymers, and combinations thereof

Where desired, the support membrane can be placed over a layer containing one or more culture components, said layer being positioned on said gel medium. It is preferred that the layer be a thin film of liquid medium or a filter paper with a liquid medium absorbed therein. Where desired, the layer can contain one or more plant growth regulators (such as abscisic acid and the like) which are not also contained in the solid gel medium.

A third improvement is an improved method for regenerating plants which comprises:

(a) culturing plant cells on a support membrane placed over a gel medium to produce embryos or shoots, wherein the improvement comprises the use of a liquid-permeable support membrane prepared from low-absorption fibers that permits the diffusion of components from the medium to the plant cells to promote the growth of the plant cells above the medium without allowing the plant cells to become embedded in the medium; and

(b) germinating the embryos to produce plants.

Where desired, the plant cells can be plated onto the support membrane prior to culturing. It is preferred that the liquid-permeable support membrane be prepared from material selected from the group consisting of polyesters, polypropylenes, liquid-permeable fluoropolymers, and combinations thereof.

Where desired, the support membrane can be placed over a layer containing one or more culture components, said layer being positioned on said gel medium. It is preferred that the layer be a thin film of liquid medium or a filter paper with a liquid medium absorbed therein.

A further improvement associated with the methods of the present invention is the utility of transferring a liquid suspension plant tissue culture to a gelled medium or to a fresh liquid medium via the use of a polyester, polypropylene or liquid-permeable fluoropolymer membrane as a filter through which the liquid medium is separated from the plant tissue culture, thereby allowing the plant tissue culture to be transplanted to the other medium. It has also been found that the use of polyester, polypropylene or liquid-permeable fluoropolymer support membranes greatly facilitates the transfer of cells from one gelled medium to another. As has been previously taught for filter papers or nylon support membranes, cell suspensions can be poured over polyester, polypropylene or liquid-permeable fluoropolymer support membranes so that the membrane mesh retains the cells for removal of the liquid medium by vacuum, gravity, or capillary action. This method significantly reduces cell carryover by enabling the rapid and complete release of the cells from the former medium before placement into the subsequent medium. However, in this improvement over the use of filter papers or nylon support membranes, most of the visible cells that have been cultured on a polyester, polypropylene or liquid-permeable fluoropolymer support membranes over gelled medium can be rapidly, completely, and easily removed from the gelled medium into a liquid medium simply by dipping the support membrane, bearing tissue, into the liquid medium (thereby releasing the cells into suspension). Experiments have shown that it is significantly easier to remove callus or embryogenic cells from wetted polyester, polypropylene or fluoropolymer liquid-permeable support membranes than it is to remove such cells from solid supports made of filter paper or nylon membrane, or from the surface of semi-solid gelled media. Thus, the employment of polyester, polypropylene, or fluoropolymer liquid-permeable membranes in the noted methods greatly reduces the agitation and scraping normally associated with the removal of cells from solid supports made of filter paper or nylon membrane. Fewer cells are lost or damaged when polyester, polypropylene or liquid-permeable fluoropolymer support membranes are employed, thereby allowing a greater recovery of viable cells.

The present invention is generally useful for improving the growth of pine embryogenic cultures on gelled media. Likewise, the present invention is also applicable to plant tissue cultures from other species wherein cells might suffer detriment due to becoming partially embedded in gelled media, such as leaf, petiole, hypocotyl or stem segments, cell suspensions, friable calli, tissue homogenates, embryogenic tissue, embryo cultures, and nodule cultures.

The present invention is further useful for improving the exposure of pine embryogenic cultures to other organic compounds, such as eradicants, polymers, and plant growth regulators. Likewise, the present invention may also be applicable to exposure of plant tissue cultures from other species, such as leaf, petiole or stem segments, calli, tissue homogenates, embryogenic tissue, and nodule cultures, to such compounds, wherein cells might be unevenly exposed to these compounds if they are presented in gelled media, or wherein the compounds may not be successfully incorporated into gelled media or may be limited in diffusion into the plant cells from gelled media due to the presence of adsorption barriers or the like.

A fourth improvement is an improved method for selecting genetically transformed plant cells which comprises:

(a) culturing plant cells which have been subjected to transformation on a support membrane placed over a gel medium, wherein the improvement comprises the use of a liquid-permeable support membrane prepared from material selected from the group consisting of polyesters, polypropylenes, fluoropolymers, and combinations thereof;

(b) contacting said plant cells with a selection agent; and

(c) selecting transformed plant cells.

Where desired, the selection agent can be contained in the gel medium. Alternately, the support membrane can be placed over a layer containing the selection agent, wherein said layer is positioned on said gel medium. It is preferred that the layer be a thin film of liquid medium or a filter paper with a liquid medium absorbed therein.

Experiments were conducted to determine whether selection agents (such as kanamycin, GENETICIN®, herbicides, and the like) would be able to pass from the underlying medium through non-cellulosic support membranes to tissue in contact with the membrane. Our results indicate that the selection agents kanamycin, GENETICIN® and various herbicides of interest were able to pass through polyester support membranes, selecting tissue which has been transformed with a kanamycin, GENETICIN® or herbicide resistance gene by killing tissue which has not been transformed with this gene. Indeed, the incidence of “escapes” (i.e., untransformed cells which fail to be killed by the selection agent) was found to be lower when polyester support membranes were employed in the selection method than when the cells are cultured directly on the surface of media containing the selection agent, or when filter paper or nylon supports were used. As mentioned above, experiments with organic dyes demonstrated that passage through polyester membranes from the underlying medium into the tissue above the membrane is faster than through nylon membranes. Thus the improved results obtained via use of the present selection method may in part be due to an improved flow of selection agent through the polyester support membrane. Unlike nylon or cellulose, polyester fibers do not swell appreciably when wetted, regardless of mesh size and weave type. The improved results obtained via use of the present selection method may also be due in part to a decrease in the appearance of necrotic clumps of cells directly adjacent to the medium (which allows the selection agent to reach more of the growing cells unimpeded).

Furthermore, it is easier to disperse the callus or tissue more evenly on the surface of the support membrane using the improved method than it is to disperse the cells without partially embedding them on gelled media. The ability to grow the cells at lower densities on selection and/or to utilize a thin layer of culture tissue, rather than the relatively thick layers or clumps associated with the use of traditional selection methods, increases the likelihood that most or all cells will be exposed to the selective agents. Moreover, resuspension of callus-type or embryogenic cells in controlled volumes (e.g., in order to replate at lower density) is also facilitated because the cells are easily dispersed from the polyester, polypropylene, or fluoropolymer fabric into liquid media, and easily captured on polyester, polypropylene or fluoropolymer liquid-permeable membranes over a Buchner funnel for replacement on fresh gelled media.

The previously described experiments with organic dyes showed that relatively large molecules (such as antibiotics, multimeric sugars, and plant growth regulators) are able to pass through polyester, polypropylene, or fluoropolymer liquid-permeable support membranes (but not as rapidly through nylon support membranes) from underlying gelled media into cells cultured on the membranes. For some of these molecules in some plant culture systems, it is not desirable to incorporate them into gelled media for reasons such as heat lability, slow diffusion through gelled media, or other osmotic effects. Accordingly, a preferred improved plant cell tissue culture method (a “support membrane bi-layer” system) for plant cell culture comprises the application of such molecules in a thin film of liquid medium on top of the gelled support medium under the polyester, polypropylene or liquid-permeable fluoropolymer support membrane, or in liquid absorbed in a layer of filter paper between the gelled medium and the support membrane, thereby allowing the molecules of interest (such as growth regulators) to pass through the support membrane into the cultured cells. The liquid medium used to incorporate the compounds of interest in this improved method is identical in composition to the gelled medium on which the plant tissue is grown, except that the gelling agents and any adsorbing components (such as activated charcoal) are omitted, and components of interest for better permeation into the cells may be added. Examples may include additional growth regulators, nutrients or polymers, or higher concentrations of any of these than can be administered in the gelled media. This improved culture method using support membranes over the gelled and liquid phases can be employed with any tissue culture phase or step that does not involve the formation of roots into the culture medium. For example, the tissue culture method can be employed with a selection medium, a maintenance medium, a proliferation medium, an elongation medium, a development medium, a maturation medium, or a regeneration medium.

Accordingly, a preferred improved plant cell tissue culture method (a “bi-layer” system) for selection of transformed cells comprises the application of molecules such as antibiotics, herbicides, or other selective agents in a thin film of liquid medium on top of the gelled support medium under the polyester, polypropylene or liquid-permeable fluoropolymer support membrane, or in liquid absorbed in a layer of filter paper between the gelled medium and the polyester, polypropylene or fluoropolymer liquid-permeable support membrane, thereby allowing the molecules of interest to pass readily through the support membrane into the cultured cells. Selection agents can be heat-labile at the temperatures required for polymerization of gelled media. Thus, an additional advantage to this method is that the selection agents, added in liquid media that can be filter-sterilized, are not subjected to the temperatures used to sterilize and polymerize gelled media. The liquid medium used to incorporate the compounds of interest in this improved method is identical in composition to the gelled medium on which the plant tissue is grown, except that the gelling agents and any adsorbing components (such as activated charcoal) are omitted, and components of interest for better permeation into the cells may be added. Examples may include antibiotics, herbicides, or other selective agents, or higher concentrations of any of these than can be administered in the gelled media. This improved culture selection method can be employed with any tissue culture phase or step that does not involve the formation of roots into the culture medium. For example, the improved selection method can be employed with a maintenance medium, a proliferation medium, an elongation medium, a development medium, a maturation medium, or a regeneration medium.

A preferred improved tissue culture bi-layer selection method is to apply selection agents in a thin film of liquid medium on top of the gelled support medium and under the polyester, polypropylene or liquid-permeable fluoropolymer support membrane, or in liquid absorbed in a layer of filter paper between the gelled medium and the polyester, polypropylene or liquid-permeable fluoropolymer support membrane, thereby allowing the selection agents to pass through the polyester, polypropylene or liquid-permeable fluoropolymer support membrane into the cultured cells. The liquid medium used to incorporate the selection agents is identical in composition to the gelled medium on which the plant tissue is grown, except that gelling agents and adsorbing components are omitted, and components of interest for better permeation into the cells may be added. Examples may include antibiotics, herbicides, or other selective agents, or higher concentrations of any of these than can be administered in the gelled media. This improved culture selection method can be employed with any tissue culture phase or step that does not involve the formation of roots into the culture medium. For example, the improved selection method can be employed with a maintenance medium, a proliferation medium, an elongation medium, a development medium, a maturation medium, or a regeneration medium.

A fifth improvement is an improved method for eradicating a bacterial contaminant or a fungal contaminant from plant cells which comprises:

(a) culturing plant cells on a support membrane over a layer containing an eradicant, wherein the improvement comprises the use of a liquid-permeable support membrane prepared from material selected from the group consisting of polyesters, polypropylenes, fluoropolymers, and combinations thereof, and wherein said layer positioned over a gel medium; and

(b) recovering plant cells from which said bacterial contaminant or fungal contaminant has been eradicated.

It is preferred that the layer be a thin film of liquid medium which contains eradicant or a filter paper with a eradicant-containing liquid medium absorbed therein.

Experiments have shown that the method is applicable for the eradication of fungus from Eucalyptus callus cultures, and a mycobacterium and a eubacterium from pine embryogenic cultures. Likewise, the present invention is also applicable to eradication of microbial contaminants in plant tissue cultures from other species, such as leaf, petiole or stem segments, calli, tissue homogenates, embryogenic tissue, and nodule cultures, wherein tissues might be unevenly exposed to the eradication agent if the eradication agent is presented in gelled media. The present invention is also applicable can also be used to eradicate many types of contaminants, such as other bacteria or fungi, and it can be used to de-contaminate many types of plant tissue cultures, such as small explants and other plant material that can form suspensions (e.g. friable calli, embryogenic tissue, nodule cultures and the like).

The improved method for eradicating contaminants from tissue cell cultures comprises application of eradicants (such as carbenicillin, ticarcillin, cefotaxime, and the like) in a thin film of liquid medium on top of the gelled support medium under the polyester, polypropylene or liquid-permeable fluoropolymer support membrane, or in liquid absorbed in a layer of filter paper between the gelled medium and the support membrane, thereby allowing the eradicants to pass through the support membrane into the cultured cells. This method can be used in addition to, or instead of, the stringent washing methods described below. The liquid medium used to incorporate the eradicants is identical in composition to the gelled medium on which the plant tissue is grown, except that gelling agents and adsorbing components are omitted, and components of interest for better permeation into the cells may be added. Examples may include antibiotics, herbicides, or other selective agents, or higher concentrations of any of these than can be administered in the gelled media may be added. This improved method of eradicating microbial contaminants from plant tissue cultures can be employed with any tissue culture phase or step that does not involve the formation of roots into the culture medium. For example, the improved eradication method can be employed with a maintenance medium, a proliferation medium, an elongation medium, a development medium, a maturation medium, or a regeneration medium.

Media components such as eradicants, plant growth regulators, and selection agents can also constitute much of the cost of tissue culture. Thus, another advantage to our methods lies in the small volume of liquid medium that is required to apply the component of interest. For example, the plant tissue may be grown on the surface of 20-30 ml of gelled medium in a petri dish, but only a few milliliters of overlaying liquid eradicant medium at the same concentration is necessary to restrict growth of microbial contaminants. The liquid medium, rather than beading up as liquid might on the surface of a glass or plastic plate, spreads over the surface of the plant tissue and gelled medium by simple surface tension. Therefore, only a fraction of the amount of eradicant need be employed in the improved bi-layer tissue culture method. A similar component reduction can be made when using the bi-layer method with selection agents such as GENETICIN® or plant growth regulators such as abscisic acid.

The flexibility of the bi-layer liquid eradicant surface application method may allow even more savings in eradicant. Unlike gelled media (which must often be made fresh some days before needed and in which the eradicants often have a short half-life), aliquots of liquid eradicant-containing media can be frozen almost indefinitely for use when required. Furthermore, after a transfer onto fresh gelled medium lacking incorporated eradicant, the cultures which would still suffer microbial regrowth are readily distinguishable from those which have already undergone sufficient eradication, whereas with eradicant incorporated in the medium these will not be distinguishable. If it can be determined which cultures are no longer contaminated, the eradicant that would have been used for them is spared; while a liquid eradicant overlay can be added without significant delay to those cultures requiring it. Though the invention described here has been tested particularly in transformation of pine somatic embryogenic cultures and tobacco suspension cultures by Agrobacterium, it can also be used for eradication with Agrobacterium transformation of many types of small explants or callus of any species that can be shown to grow on polyester, polypropylene or liquid-permeable fluoropolymer membranes over gelled media. The method can also be applied to eradication of other types of recurring microbial contaminants from valuable cultures.

Thus, a further subject of the present invention is method for eradicating a bacterial contaminant or a fungal contaminant from plant cells which comprises:

(a) washing contaminated plant cells in a liquid wash medium;

(b) plating said cells on a liquid-permeable support membrane prepared from material selected from the group consisting of polyesters, polypropylenes, fluoropolymers, and combinations thereof;

(c) recovering decontaminated plant cells with minimal physical damage.

It is preferred that steps (a) and (b) be repeated until microbial contamination is no longer detectable (commonly from 2 to 10 times). Where desired, the plant cells can be plated onto the support membrane prior to culturing.

It is preferred that the liquid wash medium contain one or more eradicants selected from the group consisting of antibiotics, bacteriostatic agents, antifungal agents, fungistatic agents, and combinations thereof.

It is further preferred that each wash be carried out for a duration sufficient to expose all the plant cells to the wash medium without interfering with subsequent growth of the plant cells (commonly from a few minutes to overnight in duration).

A further subject of the present invention is a method for eradicating a bacterial contaminant or a fungal contaminant from plant cells which comprises:

(a) washing contaminated plant cells in a liquid wash medium for a duration sufficient to expose all the plant cells to the wash medium without interfering with subsequent growth of the plant cells (commonly from a few minutes to overnight in duration);

(b) plating said plant cells on a liquid-permeable support membrane prepared from material selected from the group consisting of polyesters, polypropylenes, fluoropolymers, and combinations thereof;

(c) culturing said plant cells on said support membrane over a layer containing an eradicant, wherein said layer is positioned over a gel medium; and

(d) recovering plant cells from which said bacterial contaminant or fungal contaminant has been eradicated.

It is preferred that steps (a) and (b) be repeated until microbial contamination is obviously considerably decreased (commonly from 2 to 10 times).

It is preferred that the layer be a thin film of liquid medium or a filter paper with a liquid medium absorbed therein.

Where desired the plant cells can be plated onto the support membrane prior to culturing.

It has also been found that the employment of polyester, polypropylene or liquid-permeable fluoropolymer support membranes is beneficial during these stringent washing procedures. In such washing procedures the cells are transferred from the medium in or on which they were inoculated into wash medium, then repeatedly transferred by resuspension into fresh aliquots of this liquid medium. Incomplete transfer of cells from the semi-solid medium surface (which often results in loss of many of the possibly transformed cells) is greatly reduced through the use of polyester supports. Moreover, because the resuspension and replating of the cells is much faster, more successive wash steps and more stringent washing can be done in the same period of time. Experiments were conducted wherein pine embryogenic cells, which had been washed by our improved method employing polyester support membranes, were replated onto medium that did not contain antibiotics. No significant contamination of the replated pine cells by the bacterium with which they had previously been infected was observed in these experiments. The experiments were repeated with three different strains of the same bacterium including a hypervirulent strain. We also demonstrated that this improvement to Agrobacterium transformation protocols could also be useful in eradicating other types of bacterial and fungal contaminants in situations where the plant material is too valuable to be lost.

A sixth improvement is an improved method for producing transgenic plants which comprises:

(a) culturing plant cells which have been subjected to transformation on a support membrane placed over a first gel medium, wherein the first improvement comprises the use of a liquid-permeable support membrane prepared from material selected from the group consisting of polyesters, polypropylenes, fluoropolymers, and combinations thereof;

(b) contacting said plant cells with a selection agent;

(c) selecting transformed plant cells;

(d) promoting the production of embryos or shoots from said transformed plant cells by culturing the transformed plant cells on a second support membrane placed over a gel medium, wherein the second improvement comprises the use of a liquid-permeable second support membrane prepared from low-absorption fibers that permits the diffusion of components from the medium to the plant cells to promote the growth of the plant cells above the medium without allowing the plant cells to become embedded in the medium, and

(e) rooting said shoots or germinating said embryos to produce transgenic plants.

It is preferred that the second liquid-permeable support membrane be prepared from material selected from the group consisting of polyesters, polypropylenes, fluoropolymers, and combinations thereof.

Where desired, the selection agent can be contained in the first gel medium. Alternately, the support membrane can be placed over a layer containing the selection agent, wherein said layer is positioned on the first gel medium. It is preferred that the layer be a thin film of liquid medium or a filter paper with a liquid medium absorbed therein.

Where desired, the transformed plant cells can be plated onto the second support membrane prior to culturing. Where desired, the second support membrane can be placed over a layer containing one or more culture components, said layer being positioned on said second gel medium. It is preferred that the layer be a thin film of liquid medium or a filter paper with a liquid medium absorbed therein.

The present invention is also useful for improving selection of transformed cells by exposure of the embryogenic cultures to selection agents (e.g. antibiotics and herbicides), following the application of a genetic transformation protocol, of which many are known to those skilled in the art, including but not limited to transformation by Agrobacterium or particle-bombardment (such as BIOLISTICS® and the like). Likewise, the present invention is also applicable to selection following transformation of plant tissue cultures (such as leaf, petiole or stem segments, calli, tissue homogenates, embryogenic tissue, and nodule cultures) wherein cells might be unevenly exposed to the selection agent if the selection agent is presented in gelled media.

The following examples are provided to further illustrate the present invention and are not to be construed as limiting the invention in any manner. Standard techniques well known in the art or the techniques specifically described below are utilized.

### EXAMPLE 1

This example teaches a method for improving the growth and proliferation of callus cultures of diverse angiosperm species, and teaches a method to improve the differentiation of callus cultures to form regenerated shoots. In particular, this example teaches a method of spreading callus tissues or suspension cell cultures derived from callus tissues, from diverse angiosperm species comprising both herbaceous and woody plants, onto support membranes, which can be placed on growth or callus proliferation media and transferred from one plate of medium to the next to facilitate transfer of the cells borne on the support membranes with no loss of cells in the medium.

Cottonwood (Populus sp.) callus cultures had been generated by cutting healthy petioles from in vitro shoot cultures and placing the explants onto PHYTAGAR®-gelled DKW medium with DCR vitamins, 0.1 g/L myo-inositol, 3% sucrose, 1 mg/L BAP, and 1 mg/L 2,4-D, in 100×25 mm sterile plastic petri dishes. Cottonwood suspension cell cultures had been generated by breaking up the resulting callus into liquid medium that had the same formulation except that no agar was added, and agitating the suspensions in 250 ml Nephelo sidearm flasks at 100 rpm in a 12-hour day, 12-hour night cycle at 27±3° C.

Tobacco (Nicotiana tabacum) callus cultures had been generated by cutting healthy leaves from in vitro shoot cultures and placing the explants onto agar-gelled MS medium with Gamborg's B5 vitamins, 3% sucrose, and 2 mg/L 2,4-D, in 100×25 mm sterile plastic petri dishes. Tobacco suspension cell cultures had been generated by gently breaking up the resulting callus into liquid medium that had the same formulation except that no agar was added, and agitating the suspensions in 125 ml sidearm flasks at 100 rpm in a 12-hour day, 12-hour night cycle at 27±3° C.

It will be understood by those skilled in the art of plant tissue culture that many different temperatures, lighting conditions, media, gelling agents, and plant growth regulator concentrations could be used with the present method. As examples, other salt formulations such as WPM (Woody Plant Medium) may be substituted for MS or DKW, and other cytokinins, such as zeatin or kinetin, could be substituted for BAP.

The treatments that were used in this experiment were as follows:

A: cells maintained directly on the surface of gelled medium, and

B: cells maintained on woven polyester support membranes (PECAP® with a 35 micron pore size, Catalog No. 7-35/11 from Sefar) on the surface of gelled medium.

At the start of this experiment, six replicate plates of tobacco tissue and six replicate plates of cottonwood tissue were generated for each of the treatments. For each plate, a sterile 55×55 mm square polyester membrane support was placed in a sterile Buchner funnel.

For cottonwood, 2.5 ml of suspension culture cells and medium were pipetted onto each membrane support, and then the liquid medium was suctioned from the cells using a mild vacuum. For plates in treatment A, the cells were then gently removed from the membrane support onto a plate of agar-solidified DKW medium, of the same formulation as listed above. For plates in treatment B, each membrane support with cells was removed from the Buchner funnel and placed directly onto a plate of the same medium. The cells, or membrane supports bearing the cells, were then weighed. Dishes were incubated in a growth chamber in a 12-hour day, 12-hour night cycle at 27±3° C. They were re-weighed bi-weekly and then transferred to new petri dishes containing fresh medium.

For tobacco, 0.4 ml of suspension culture cells and medium were pipetted onto each-membrane support, and then the liquid medium was suctioned from the cells using a mild vacuum. For plates in treatment A, the cells were then gently removed from the membrane support onto a plate of agar-solidified MS medium containing Gamborg's B5 vitamins, 3% sucrose, and 2 mg/L 2,4-D. For plates in treatment B, each membrane support with cells was removed from the Buchner funnel and placed directly onto a plate of the same medium. The cells, or membrane supports bearing the cells, were then weighed. Dishes were incubated in a growth chamber in a 12-hour day, 12-hour night cycle at 27±3° C. They were re-weighed weekly, and transferred to new petri dishes containing fresh medium every 3 weeks.

Although the starting weights of the callus placed in each of the two treatments were equivalent within each species, the growth rates differed between the two treatments, as was apparent in each subsequent weighing. Data for cottonwood are shown in Table 1, and data for tobacco are shown in Table 2.

TABLE 1
Growth Rate of Cottonwood Callus Plated Directly On Gelled Medium
Or Over A Support Membrane

| | Average growth³ | | |
|---|---|---|---|
| | First | Second | Third |
| | two-week period | two-week period | two-week period |
| Treatment A¹ | 0.05 ± 0.03 | 0.23 ± 0.13 | 1.12 ± 0.66 |
| Treatment B² | 0.15 ± 0.07 | 0.60 ± 0.29 | 2.22 ± 1.08 |

¹A = cells maintained directly on the surface of gelled medium.
²B = cells maintained on polyester support membranes on the surface of gelled medium.
³Average growth in grams per plate during each period.

It was necessary to weigh and transfer the cottonwood callus less often than tobacco callus due to generally slower growth of woody plant callus. However, the growth rate of the callus grown on support membranes over the gelled medium was in every weighing period greater than double the growth rate of the callus maintained directly on the gelled medium. Within four weeks it could be seen readily that the callus maintained on the membranes was more friable and greenish yellow in color, compared to the clumpy, tanner callus growing directly in the medium. The coloration difference may be attributed to production of anthocyanin pigments and phenolics by tissue that is under greater stress.

TABLE 2
Growth Rate Of Tobacco Callus Plated Directly
On Gelled Medium Or Over A Support Membrane

| | Average growth³ | | | | |
|---|---|---|---|---|---|
| | Week 1 | Week 2 | Week 3 | Week 4 | Week 5 |
| Treatment A¹ | 0.72 ± 0.165 | 1.07 ± 0.177 | 1.75 ± 0.566 | 2.88 ± 1.366 | 2.70 ± 2.000 |
| Treatment B² | 1.54 ± 0.270 | 3.21 ± 0.516 | 3.24 ± 0.872 | 6.37 ± 1.651 | 3.74 ± 1.301 |

¹A = cells maintained directly on the surface of gelled medium.
²B = cells maintained on polyester support membranes on the surface of gelled medium.
³Average growth in grams per plate during each period.

The growth rate of the tissue in the treatment placed on polyester support membranes was greater than double that of the tissue placed directly on the medium throughout most of the period of observation. The growth rate slowed, but was still nearly double, in the fifth week as the plate became nearly completely overgrown with plant tissue. The callus on the support membrane plates seem lighter-colored and more dry and fluffy compared to the callus maintained directly on the surface of the medium, which had a darker color (perhaps due to stress-related release of phenolics) and appeared more water-soaked and slushy.

Unlike the cottonwood callus, this line of tobacco callus is able to regenerate shoots when placed on medium containing appropriate levels of BAP and lacking 2,4-D. Accordingly, following this period of growth, 2 g tobacco tissue from each replicate plate of each treatment was placed onto regeneration medium in order to determine whether the rapid growth or the altered physical contact may have affected the health, vigor, and regenerability of the callus and its ability to take up an altered growth regulator complement from the medium. The regeneration medium used was identical to that used for callus induction and maintenance, except that 1 mg/L BAP was substituted for the 2,4-D. Tissue (1 g) from each replicate of each treatment was placed directly onto the surface of regeneration medium, and an additional 1 g tissue from each replicate of each treatment was placed on a polyester support membrane laid on the surface of the regeneration medium. This cross-treatment served to indicate whether growth on the support membranes followed by a switch to no membrane, or vice versa, might have an effect on regeneration potential. Petri dishes containing the callus regeneration treatments were incubated in a growth chamber in a 12-hour day, 12-hour night cycle at 27±3° C., and examined with a stereomicroscope after 3 and 8 weeks to record the presence of any shoot primordia or shoots. Data are shown in Table 3.

TABLE 3
Shoot Morphogenesis From De-Differentiated Tobacco Callus

| Treatment¹ | | Three Weeks² | | Eight Weeks³ | |
|---|---|---|---|---|---|
| Growth | Regeneration | | | | |
| medium | medium | Primordia⁴ | Shoots⁵ | Primordia⁴ | Shoots⁵ |
| A | A | 0.50 | 0.00 | 0.83 | 0.83 |
| A | B | 0.67 | 0.33 | 1.00 | 1.00 |
| B | B | 0.83 | 0.33 | 1.00 | 1.00 |
| B | A | 0.67 | 0.17 | 1.00 | 0.83 |

¹A = cells maintained directly on the surface of gelled medium.
B = cells maintained on polyester support membranes on the surface of gelled medium.
²Observation at three weeks after placement on regeneration medium.
³Observation at eight weeks after placement on regeneration medium.
⁴Average number of shoot primordia per plate (n = 6).
⁵Average number of shoots per plate (n = 6).

The three-week data show that the most rapid differentiation of shoot primordia and shoots occurred on the treatment in which both pre-regeneration growth and shoot regeneration took place on support membranes laid on the surface of the media. The eight-week data show that no treatment in which the support membranes were used at either stage was detrimental to shoot differentiation. The treatment that lagged the furthest in shoot differentiation was the sole treatment in which no support membranes were used, in either the pre-regeneration growth stage or on regeneration medium.

### EXAMPLE 2

This example teaches a method for improving the growth and proliferation of nodule-type cultures generated by many woody plants, and teaches a method to improve the organogenic differentiation of nodule cultures, in this case to form both shoots and aerial roots. Those skilled in the art of plant tissue culture will recognize from the combination of this example and the preceding one that the improved method would be applicable to a variety of tissue types, species, and regeneration protocols via organogenesis of shoots or roots. In particular, this example teaches a method of using support membranes over regeneration media to accelerate the de novo regeneration of organs from less differentiated nodule cultures.

Sweetgum (Liquidambar styraciflua) nodular cultures had been generated at the edges of in vitro shoot cultures on gelled DKW medium. Nodules were taken from the base of the shoots by cutting them off with sterile surgical scissors and placing them onto DKW medium containing 5 uM 2,4-D and 5 uM BAP, in 100×25 mm sterile plastic petri dishes. These cultures were maintained in a 12-hour day, 12-hour night cycle at 27±3° C. Six nodular pieces of similar weight and size were placed on each treatment (4 treatments total). Growth was measured by weighing the nodular pieces. The nodular pieces were pushed into the medium for Treatment A (no support membrane). For treatments B and C (35 micron pore size polyester and nylon support membranes, PECAP® and NITEX® from Sefar, respectively) the nodular pieces were placed directly on the membrane in the center. Initially it proved difficult to maintain the rounded nodular pieces on the membranes, as they continually rolled off. After several weeks it was decided to cut the nodular pieces into half-spheres, laying the cut sides down on the medium or membranes in all treatments. Recovery from the cut injury, denoted by resumed growth, required several weeks. Measurements were recorded, then, after a total of six weeks following the start of the treatments, to allow for the observation, cutting, and recovery. After this point, the tissue was transferred to fresh media and weights were recorded every three weeks. After three periods of growth were measured, nodular pieces were placed on the respective membrane support treatments over regeneration (WPM) medium, or directly on the medium in the control treatment A, in MAGENTA® boxes. All the material was examined under a dissecting microscope. Growth after the recovery period is noted in Table 4, and regeneration frequency (differentiation of roots and shoots) after the placement on regeneration medium, is noted in Table 5.

TABLE 4
Effect Of Support Membrane On Average Growth (n = 6)
Of Cut Nodule Pieces Of Sweetgum

| Pre-regeneration | Period | Period | Period |
|---|---|---|---|
| growth of nodules | One¹ | Two² | Three³ |
| cells maintained directly on | 0.22 ± 0.12 | 0.11 ± 0.09 | 0.43 ± 0.24 |
| the surface of gelled medium | | | |
| cells maintained on poly- | 0.28 ± 0.07 | 0.44 ± 0.14 | 1.37 ± 0.34 |
| ester support membranes | | | |
| on the surface of gelled | | | |
| medium | | | |
| cells maintained on nylon | 0.25 ± 0.13 | 0.39 ± 0.12 | 1.35 ± 0.37 |
| support membranes on the | | | |
| surface of gelled medium | | | |

¹First three week measuring period.
²Second three week measuring period.
³Third three week measuring period.

It can be seen that growth is marginally faster on the polyester support membranes than on the nylon support membranes, and both are substantially greater than the growth rate of nodules laid directly on the surface of the medium, indicating that there is a benefit to raising the sweetgum nodular material out from being embedded in the medium. There was also a morphological difference observed. The nodular meristematic clumps were more friable and breakable in the boxes with support membranes than those in which the tissue was placed directly on the medium.

TABLE 5
Effect Of Support Membrane On Organogenesis Of Cut Nodule Pieces Of
Sweetgum Cultured 8 Weeks Over Regeneration Medium

| | Average number per | | | |
|---|---|---|---|---|
| | explant at 8 weeks | | Percent explants showing | |
| Organogenesis | | shoot | root proliferation | |
| on nodules | shoots | primordia | 4 Weeks | 8 Weeks |
| cells maintained directly | 1.5 | 3.5 | 0 | 0 |
| on the surface of gelled | | | | |
| medium | | | | |
| cells maintained on poly- | 3 | 12 | 17% | 20% |
| ester support membranes | | | | |
| on the surface of gelled | | | | |
| medium | | | | |
| cells maintained on nylon | 3 | 9.3 | 0 | 0 |
| support membranes on | | | | |
| the surface of gelled | | | | |
| medium | | | | |

Furthermore, there may be a benefit related to better or more rapid absorption of growth regulators through the polyester membranes than through nylon membranes, as root differentiation was accelerated on the polyester membrane treatment over regeneration medium. Shoot differentiation did not appear to be accelerated or delayed in any treatment relative to any other, but more shoots developed on explants that were maintained over support membranes, possibly because the material did not appear to develop a hard, non-penetrable surface at the interface with the medium. While the average number of elongated shoots per explant developed on the two membrane types in the eight week period was equal, the polyester membrane treatment developed marginally more additional recognizable shoot primordia at the surface of the nodule than did the nylon support treatment.

Those skilled in the art of plant tissue culture will recognize that the method applied here to a variety of tissue types, from a widely diverse array of woody and herbaceous plants on various media formulations, can be applied to any plant tissue for which a growth or development benefit would accrue when the tissue is grown over any similar medium but for which a growth or development detriment results when the tissue becomes partially submerged in or embedded in the medium.

### EXAMPLE 3

This example teaches that the method also improves the maintenance and growth of embryogenic tissues. In particular, this example teaches a method of spreading embryogenic tissues on polyester support membranes, which was demonstrated to accelerate the growth and improve the maintenance efficiency of these embryogenic tissues.

Loblolly pine (Pinus taeda) and loblolly hybrid embryogenic cell lines were initiated according to the method described in Becwar et al. 1996 except that WV5 salts (Coke 1996) were used as the basal initiation medium. Media formulations used for all loblolly pine and loblolly hybrid examples contained herein are listed in Table 6, below. Those skilled in the art of plant tissue culture will recognize that many other formulations, sterilization conditions, and media volumes would be applicable to the use of the present method.

TABLE 6
Initiation, Maintenance, And Proliferation Media Formulations
Used For Pine Embryogenesis

| | Gelled | Gelled | Gelled | Liquid^{f} |
|---|---|---|---|---|
| | Initiation | Initiation | Maintenance | Proliferation |
| | Medium | Medium | Medium | Medium |
| COMPONENT | WV5₁^{a} | DCR₁ | DCR₂ | DCR₃ |
| Basal medium^{a} | WV5 | DCR | DCR | DCR |
| | CONCENTRATION (g/L) | | | |
| Inositol | 0.5 | 0.5 | 0.5 | 0.5 |
| Casein hydrolysate^{b} | 0.5 | 0.5 | 0.5 | 0.5 |
| L-glutamine | 0 | 0.25 | 0.25 | 0.25 |
| Sucrose | 0 | 30 | 30 | 30 |
| Maltose | 30 | 0 | 0 | 0 |
| Polyethylene glycol | 0 | 0 | 0 | 0 |
| GELRITE^{c} | 1.5 | 1.5 | 2 | 0 |
| Activated Carbon | 0 | 0 | 0 | 0-0.5 |
| PLANT GROWTH | CONCENTRATION (mg/L) | | | |
| REGULATORS | | | | |
| Auxin^{d} | 1.0-3.0 | 3 | 3 | 3 |
| Cytokinin^{e} | 0.5 | 0.5 | 0.5 | 0.5 |
| Abscisic Acid | 10 | 0-30.00 | 0-30.00 | 0 |

^{a}According to Coke (1996).
^{b}In some Examples, defined amino acid mixtures were substituted for casein hydrolysate.
^{c}GELRITE ® (gellan gum manufactured by Merck, Inc.).
^{d}2,4-dichlorophenoxyacetic acid (2,4-D) or naphthalene acetic acid (NAA).
^{e}N⁶-benzylaminopurine (BAP) or N⁶-benzyladenine (BA).
^{f}For all liquid culture media used in these examples, no gelling agent was added and the medium was stored in 500 ml batches under refrigeration or frozen prior to use.

The first 200 proliferating culture lines were selected for use in this study and randomly assigned to one of two treatments. The treatments define the method of maintenance:

A: Tissue lifted from the initiation medium using sterile Dumont forceps was placed directly onto the surface of the gelled maintenance medium.

B: The method uses 55×55 mm square polyester membranes (with edges “pinked” or sealed when cut by laser to prevent fraying) as an interface between the callus cells and the gelled medium. Tissue lifted from the initiation medium using sterile Dumont forceps was placed onto a polyester membrane with a 35 micron pore size (Sefar PECAP® Catalog No. 7-35/11) laid on the surface of the gelled maintenance medium.

The cultures were incubated in the dark at a constant temperature of 23° C.±2° C. When each culture reached a minimum size (approximately 0.3-0.5 g), or at least every two weeks whether it had reached that size or not, it was subcultured by transferring it to fresh maintenance medium (with a fresh polyester membrane, for Treatment B). Cells that stuck to or were embedded in the surface of the media could not be transferred. Any obviously necrotic cells were also discarded. Minor culture loss due to contamination resulted in a total of 96 lines being evaluated for Treatment A, and 98 lines being evaluated for Treatment B. Beginning four weeks after start of the experimental treatments, cultures were examined weekly and data taken on the number that had reached a target mass of at least 2 grams. When cultures reached a total cell mass of at least 2 grams, or when cultures were discarded for reasons of culture decline, the date was recorded. The experiment was terminated after 10 weeks (a total of 18 weeks after the date of explant).

There was a significant treatment effect on the number of lines that grew to a total mass of at least 2 grams within 18 weeks from explant plating (Table 7). In the treatment using polyester support membranes (Treatment B), 39 of 98 lines grew to at least 2 grams, while only 17 of 96 lines growing directly on the gelled medium reached 2 grams during the 18-week time period.

TABLE 7
Number Of Embryogenic Cultures That Grew To At Least 2 g
Over The Course Of 18 Weeks From Explant, Using Each Of
Two Maintenance Methods

| Maintenance Method | Starting Cell Lines | Cell Lines reaching 2 g |
|---|---|---|
| Directly on medium | 96 | 17 |
| On polyester support | 98 | 39 |

More cultures were successful (attained a mass of 2 g) when maintained on polyester support membranes because the tissues grew more quickly than those maintained directly on the surface of the media. The data in Table 8 show how many, and when, cultures attained a mass of at least 2 grams for the two maintenance methods, beginning at 12 weeks from explant plating.

TABLE 8
Number Of Embryogenic Cultures Attaining A Mass
Greater Than 2 g At Number Of Weeks From Explant.

| | Number of Cultures Attaining | | | | |
|---|---|---|---|---|---|
| Maintenance | 2 g by Week From Explant Plating | | | | |
| Method | Week 12 | Week 13 | Week 14 | Week 15 | Week 18 |
| Directly on | 0 | 2 | 7 | 5 | 3 |
| medium | | | | | |
| surface | | | | | |
| On polyester | 12 | 12 | 2 | 13 | 0 |
| support | | | | | |
| membrane | | | | | |

A comparison of how many cultures grew to 2 grams at each week after explant plating is not as important as the difference in the time periods involved for the two methods (Table 8). Twelve cultures from the polyester support method grew to 2 grams by 12 weeks from explant plating, and the remaining cultures that did reach 2 grams did so by week 15. In contrast, only two cultures from the control method attained 2 grams by week 13. Most that reached 2 grams did so by week 14, but some took the entire 18 weeks from explant plating to grow sufficiently.

To summarize, there was a two-week difference as to when the first significant numbers of cultures reached 2 grams between the two methods. In addition, the number of weeks between the first and last cultures attaining 2 grams was less for cultures maintained on polyester support membranes (3 weeks) than for those maintained directly on the surface of the medium (5 weeks).

We observed that cells are very easily removed from the surface of polyester support membranes without sticking. The cells could then be dispersed on a fresh membrane in a thin layer by physical manipulation with the forceps (the degree to which the cells can be dispersed varies slightly from line to line and is dependent on the morphology of the line, particularly the moisture content). In general, on the polyester support membranes over maintenance/proliferation media, cell morphology appeared much healthier with far less necrosis present (effectively increasing the volume of viable/usable cells). Although tissue necrosis was not eliminated using the thin layer method, it was less prevalent. When maintained directly on the surface of the medium, cells in the center of the clumps often became necrotic, thereby limiting the total amount of the clump that could be subcultured. Maintenance of the tissue in a layer over the polyester support membrane reduced the amount of tissue necrosis, and therefore, a larger percentage of the tissues were vigorous.

The experiments have been repeated with a variety of embryogenic cell lines from different genetic backgrounds and at different physiological stages, maintained for different time periods and with different subculture frequencies. In every case, superior growth was observed on polyester support membranes.

### EXAMPLE 4

This example teaches further on the method for improving the growth and proliferation of embryogenic tissues, and teaches a method to improve the development of embryos to form regenerated plants from these tissues. In particular, this example teaches a method of spreading pine embryogenic tissues on polyester or ethylene tetrafluoroethylene (ETFE) support membranes, which can be transferred from one plate of medium to the next to facilitate transfer of the cells borne on the support membranes with no loss of cells in the medium. Furthermore, this example teaches an improved method of using polyester or ETFE support membranes to accelerate the growth and increase the number of embryos harvested from pine embryogenic tissues when compared to the use of nylon support membranes.

Using the methods of Example 3 (except that cell cultures from both P. taeda and a hybrid of P. rigida×P. taeda were initiated on DCR₁ initiation medium and maintained on DCR₂ maintenance medium, Table 6), cell cultures containing globular and precotyledonary embryogenic tissue were obtained. In this example, after one to three months of culture on DCR₁ semi-solid maintenance medium, the tissue cultures had been cryopreserved by methods well known to those skilled in the art of plant tissue culture. This example serves to demonstrate that the improvement obtained using the method taught in the previous example is applicable to embryogenic tissue from a variety of pre-treatments, physiological backgrounds, species and hybrids.

Following retrieval of the cells from cryopreservation for these experiments, suspension cultures were established by inoculating a 250 ml Nephelo sidearm flask (Kontes Chemistry and Life Sciences Products) with 1 g of tissue from each of three genetically different tissue culture lines into 20 ml liquid DCR₃ medium. The flasks containing the cells in liquid medium were then placed on a gyrotory shaker at 100 rpm in a dark culture room at a temperature of 23±2° C. One week later, the liquid in each flask was brought up to 35 ml by pouring 15 ml fresh medium into the culture flask and swirling to evenly distribute the cells. At 7-day intervals the cell growth was measured in the sidearm by decanting cells and medium into the sidearm portion of the flasks, allowing the cells to settle for 30 minutes and then measuring the settled cell volume (SCV). When each suspension's SCV was greater than or equal to half the maximal SCV (50% of the volume of the flask was occupied by plant cells), it was split with half going into another sidearm 250 ml flask, and both flasks were brought up to 35 ml with fresh medium. When the SCV was greater than or equal to half the maximal SCV, each culture was transferred to a 500 ml sidearm flask containing a total of 80 ml cells and medium, for routine maintenance.

For tests of the effects of different types of membranes on growth and proliferation of embryogenic cells, each of the lines was plated on DCR₂ maintenance/proliferation medium (Becwar et al. 1996) over different membrane support treatments. The treatments were as shown in Table 9. A range of fiber types (which corresponded to different liquid absorption and resistance characteristics) and mesh sizes was tested; in theory, pore sizes from 0.2 microns up to about half the size of the cells being cultured could be used, to allow permeability to liquid medium and complex organic molecules without loss of the cultured cells through the mesh. The membranes being tested were available in a range of thicknesses and displayed variation in other characteristics such as thread size and percentage open area, as shown in Table 9 below.

TABLE 9
Properties Of Membrane Supports Tested In Example 4

| | | | | | Thread | Membrane | |
|---|---|---|---|---|---|---|---|
| Treatment | | | Mesh | | diameter | thickness | SEFAR |
| code | Fiber type¹ | % open area² | opening³ | Mesh/cm⁴ | (microns) | (microns) | Catalog No.⁵ |
| A | N | 9.5 | 15 | 202 | 35 | 60 | N 3-15/10 |
| B | N | 16 | 35 | 100 × 128 | 35 | 97 | N 3-35/16 |
| C | N | 22 | 58 | 82 | 65 | 110 | N 3-58/22 |
| D | N | 30 | 63 | 71 × 94 | 43 | 95 | N 3-63/30 |
| E | N | 21 | 74 | 61 | 89 | 155 | N 3-74/21 |
| F | N | 34 | 85 | 65 × 74 | 50 | 98 | N 3-85/30 |
| G | P | 45 | 118 | 52 × 59 | 40/64 | 106 | P 7-118/45 |
| H | P | 12 | 21 | 163 | 41 | 70 | P 7-21/12 |
| I | P | 21 | 30 | 173 | 28 | 50 | P 7-30/21 |
| J | P | 21 | 35 | 150 | 33 | 65 | P 7-33/21 |
| K | P | 11 | 35 | 92 | 64 | 125 | P 7-35/11 |
| L | P | 36 | 51 | 119 | 33 | 65 | P 7-51/36 |
| M | P | 20 | 52 | 90 | 55 | 101 | P 7-52/20 |
| N | P | 33 | 59 | 97 | 44 | 65 | P 7-59/33 |
| O | P | 39.5 | 73 | 87 | 43 | 65 | P 7-73/40 |
| P | E | 27 | 85 | 61 | 80 | 157 | F 9-85/27 |

¹N = nylon fiber mesh which absorbed 3.8 to 4.0% liquid from underlying media. The mesh's resistance to citric acid was conditional.
P = polyester fiber mesh which absorbed 0.4% liquid from underlying media. The mesh's resistance to citric acid was satisfactory under all conditions.
E = ethylene tetrafluoroethylene fiber mesh did not absorb any liquid from underlying media. The mesh's resistance to citric acid was satisfactory under all conditions.
²Percent open area in dry membrane.
³Mesh opening in dry membrane.
⁴Some support types were generated using a 3:1 taffeta weave rather than a 1:1 straight or twill weave, resulting in two measurements for mesh/cm. In these experiments, as can be seen by comparison of Table 9 above with Tables 11 and 12 below, the independent effect of the weave type was not found to be significant.
⁵N = NITEX ®; P = PECAP ®; F = FLUORTEX ®.

Three replicate plates were generated from each cell line for each of the treatments. For each plate, a sterile 55×55 mm square membrane support of the type listed for the corresponding treatment was placed in a sterile Buchner funnel. Suspension culture cells and medium, measured by SCV to give an equivalent amount of cells for each cell line, were pipetted onto each membrane support. The liquid medium was then suctioned from the cells using a mild vacuum. Each membrane support with cells was removed from the Buchner funnel and placed on MSG₁ embryo development/maturation medium containing 125 mg/l ABA (Table 10 below) to assess the ability of the cultures to develop high quality harvestable cotyledonary-embryos. Dishes were incubated in a dark growth chamber at 23±2° C. The membrane supports were then transferred to new petri dishes containing fresh medium every 3 weeks. At week 9 cotyledonary embryos were counted and those deemed suitable for germination were harvested. Results are shown in Table 11 below.

TABLE 10
Composition of Development/Maturation and
Germination Media Used For Pine Embryogenic Cells

| | Development/ | | |
|---|---|---|---|
| | Maturation | Pre-Germination | Germination |
| | Medium | Medium | Medium |
| COMPONENT | MSG₁ | MSG₂ | MSG₃ |
| Basal medium^{a} | MSG | MSG | MSG |
| | CONCENTRATION (g/L) | | |
| Ammonium | 0 | 0 | 0.80 |
| Nitrate | | | |
| Inositol | 0.10 | 0.10 | 0.10 |
| L-glutamine | 1.45 | 1.45 | 0 |
| Sucrose | 0 | 0 | 30.00 |
| Maltose | 60.00 | 60.00 | 0 |
| GELRITE^{b} | 2.00 | 2.00 | 2.00 |
| Activated | 0-1.25 | 0 | 5.00 |
| Carbon | | | |
| PEG^{c} | 0-100.00 | 0 | 0 |
| PLANT GROWTH | Concentration (mg/L) | | |
| REGULATORS | | | |
| ABA^{d} | 125 | 21 | — |

^{a}According to Becwar et al. (1990).
^{b}GELRITE ® (gellan gum manufactured by Merck, Inc.).
^{c}Polyethylene glycol (molecular weight of 4000).
^{d}Abscisic acid.

At the same time, P. taeda and hybrid lines were also plated on each of the membrane support treatments over DCR₂ maintenance/proliferation medium (Table 5). Equivalent amounts of cells for each of the lines were placed onto three replicate membrane supports for each of the treatments in the same manner as described above. Each membrane support with cells was removed from the Buchner funnel and placed on DCR₂ maintenance/proliferation medium. Petri dishes were incubated in a dark growth chamber at 23±2° C. The membrane supports bearing the cells were then transferred to new petri dishes containing fresh medium every 2-3 weeks. Growth of the cells was measured in grams using a sterile milligram balance. The results are shown in Table 12 below.

After the cell masses on DCR₂ maintenance/proliferation medium had been allowed to proliferate for six weeks, they were resuspended in DCR₃ liquid medium again as described above, and re-plated on fresh membrane supports of the same treatment as used during proliferation. Three replicate plates were generated from each of two embryogenic cell lines (one P. taeda line and one hybrid pine line) for each of the treatments. When the cell suspensions had been brought to approximately identical (half-maximal) SCV, equivalent amounts of suspension culture cells were pipetted onto sterile 55×55 mm square membrane supports of the type listed for each corresponding treatment as above, for placement on MSG₁ development/maturation medium as above, to assess the ability of the cultures to develop high quality harvestable stage 3 embryos after both proliferative growth and maturation on the respective membrane treatments. Dishes were incubated in a dark growth chamber at 23±2° C. The membrane supports were transferred to new petri dishes containing fresh medium every 3 weeks. At week 9, stage 3 embryos were counted and those deemed suitable for germination were harvested. The results are shown in Table 11 below.

TABLE 11
Average Number of Embryos

| Line 1 | Treatment¹ | After Cryoretrieval² | Six Weeks³ |
|---|---|---|---|
| P. taeda | A | 76.0 ± 15.7 | 76.7 ± 46.2 |
| Hybrid | A | 183.0 ± 24.8 | 155.7 ± 26.7 |
| P. taeda | B | 57.7 ± 22.0 | 44.7 ± 11.4 |
| Hybrid | B | 110.3 ± 15.7 | 169.3 ± 7.2 |
| P. taeda | C | n.d.⁴ | 24.0 ± 6.2 |
| Hybrid | C | n.d. | 101.3 ± 20.5 |
| P. taeda | D | 173.3 ± 32.7 | 56.3 ± 31.2 |
| Hybrid | D | 137.0 ± 7.0 | 137.0 ± 18.1 |
| P. taeda | E | n.d. | 52.0 ± 33.1 |
| Hybrid | E | n.d. | 103.7 ± 90.4 |
| P. taeda | F | 190.0 ± 62.4 | 25.0 ± 13.2 |
| Hybrid | F | 157.0 ± 27.2 | 123.7 ± 14.6 |
| P. taeda | G | n.d. | 165.3 ± 29.0 |
| Hybrid | G | n.d. | 169.0 ± 33.4 |
| P. taeda | H | 141.3 ± 17.0 | 42.7 ± 9.1 |
| Hybrid | H | 132.7 ± 20.2 | 135.0 ± 23.6 |
| P. taeda | I | 173.7 ± 16.7 | 34.7 ± 4.2 |
| Hybrid | I | 119.0 ± 2.6 | 129.7 ± 9.3 |
| P. taeda | J | 203.3 ± 48.8 | 34.3 ± 7.4 |
| Hybrid | J | 116.0 ± 11.5 | 75.3 ± 20.7 |
| P. taeda | K | 88.7 ± 25.0 | 34.7 ± 9.5 |
| Hybrid | K | 113.0 ± 16.1 | 185.7 ± 27.3 |
| P. taeda | L | 74.3 ± 18.6 | 101.7 ± 9.1 |
| Hybrid | L | 122.0 ± 21.9 | 213.3 ± 25.7 |
| P. taeda | M | 90.3 ± 24.1 | 26.0 ± 6.6 |
| Hybrid | M | 102.0 ± 9.2 | 149.3 ± 28.0 |
| P. taeda | N | n.d. | 148.0 ± 64.6 |
| Hybrid | N | n.d. | 147.7 ± 13.6 |
| P. taeda | O | n.d. | 34.0 ± 58.9 |
| Hybrid | O | n.d. | 156.7 ± 33.0 |
| P. taeda | P | n.d. | 192.7 ± 58.8 |
| Hybrid | P | n.d. | 191.7 ± 14.5 |

¹Treatment according to Table V.
²Average number of embryos produced on replicate plates of tissue placed over development medium on various types of support membranes immediately after recovery from cryoretrieval.
³Average number of embryos produced on replicate plates of tissue placed over development medium on various types of support membranes after six weeks maintenance proliferation on same support membrane type recovery from cryoretrieval.
⁴n.d. = not done.

TABLE 12
Growth And Proliferation Of Embryogenic Cell Cultures
On Different Types Of Support Membranes

| | | Gain in tissue weight in grams on | |
|---|---|---|---|
| | | proliferation medium over membrane support | |
| Line¹ | Treatment² | in two weeks | in six weeks |
| L1 | A | 2.790 ± 0.763 | 7.467 ± 0.696 |
| L2 | A | 4.590 ± 1.181 | 9.833 ± 0.964 |
| H1 | A | 2.470 ± 1.310 | 7.843 ± 2.013 |
| L1 | B | 0.800 ± 0.165 | 3.637 ± 0.212 |
| L2 | B | 3.260 ± 0.452 | 9.407 ± 1.138 |
| H1 | B | 2.730 ± 0.062 | 7.850 ± 0.433 |
| L1 | C | 2.867 ± 0.283 | 6.750 ± 0.877 |
| L2 | C | 4.240 ± 1.005 | 9.507 ± 0.640 |
| H1 | C | 3.523 ± 0.316 | 9.817 ± 0.546 |
| L1 | D | 2.497 ± 0.514 | 6.583 ± 0.663 |
| L2 | D | 4.687 ± 0.465 | 8.723 ± 0.604 |
| H1 | D | 3.237 ± 0.354 | 8.910 ± 0.816 |
| L1 | E | 1.520 ± 0.296 | 4.533 ± 0.516 |
| L2 | E | 4.660 ± 0.262 | 9.760 ± 1.612 |
| H1 | E | 3.547 ± 0.214 | 9.137 ± 0.142 |
| L1 | F | 2.840 ± 0.288 | 6.557 ± 0.441 |
| L2 | F | 4.820 ± 0.377 | 9.400 ± 1.048 |
| H1 | F | 3.087 ± 0.912 | 8.687 ± 0.624 |
| L1 | G | 2.220 ± 0.265 | 5.133 ± 0.429 |
| L2 | G | 3.970 ± 0.655 | 9.537 ± 1.131 |
| H1 | G | 3.207 ± 0.420 | 7.893 ± 0.722 |
| L1 | H | 3.043 ± 0.511 | 6.247 ± 1.123 |
| L2 | H | 4.610 ± 0.560 | 10.717 ± 0.964 |
| H1 | H | 3.957 ± 0.483 | 8.913 ± 0.873 |
| L1 | I | 3.383 ± 0.581 | 6.563 ± 0.559 |
| L2 | I | 4.560 ± 1.079 | 10.557 ± 0.644 |
| H1 | I | 3.970 ± 0.205 | 8.427 ± 0.369 |
| L1 | J | 2.907 ± 0.244 | 6.537 ± 0.746 |
| L2 | J | 4.753 ± 1.193 | 10.803 ± 0.609 |
| H1 | J | 3.270 ± 1.264 | 8.640 ± 1.618 |
| L1 | K | 2.660 ± 0.400 | 5.997 ± 0.347 |
| L2 | K | 4.697 ± 0.376 | 9.607 ± 0.774 |
| H1 | K | 3.207 ± 1.018 | 9.040 ± 0.439 |
| L1 | L | 2.690 ± 0.384 | 6.250 ± 0.265 |
| L2 | L | 4.513 ± 1.366 | 10.370 ± 1.710 |
| H1 | L | 2.947 ± 0.591 | 9.177 ± 0.996 |
| L1 | M | 2.603 ± 0.286 | 6.307 ± 0.813 |
| L2 | M | 3.900 ± 0.252 | 9.477 ± 0.206 |
| H1 | M | 3.217 ± 0.324 | 9.230 ± 1.178 |
| L1 | N | 2.957 ± 0.391 | 5.967 ± 0.788 |
| L2 | N | 3.927 ± 0.903 | 9.523 ± 0.715 |
| H1 | N | 3.607 ± 0.798 | 8.163 ± 2.2025 |
| L1 | O | 3.107 ± 0.706 | 7.473 ± 1.541 |
| L2 | O | 4.590 ± 1.208 | 10.617 ± 1.267 |
| H1 | O | 3.723 ± 0.725 | 11.007 ± 0.180 |
| L1 | P | 2.213 ± 0.519 | 5.727 ± 0.595 |
| L2 | P | 4.943 ± 0.267 | 10.477 ± 0.818 |
| H1 | P | 3.247 ± 0.131 | 8.953 ± 0.934 |

¹L1 = P. taeda cell line 1; L2 = P. taeda cell line 2; H1 = hybrid pine cell line 1.
²See Table V.

Embryos harvested from the embryo development medium were again placed on membrane supports to facilitate bulk transfer of embryos through the preparatory steps for germination. The membrane supports, bearing around 25-40 harvested embryos each, were placed over gelled medium MSG₂ (Table 10), in petri plates and incubated for about four weeks in the dark at a temperature of 4° C., according to the method of Rutter et al. 1998. Next, the membrane supports still bearing the embryos were placed in sealed containers at 100% relative humidity for about three weeks in the dark at a temperature of 23±2° C., according to the method of Rutter et al. 1998. Next, the membrane supports still bearing the embryos were transferred to medium MSG₃ (Table 10) and incubated for about three days in the dark at a temperature of 27±3° C. Embryos were then removed from their membrane supports and placed individually onto the surface of fresh MSG₃ medium in petri plates for germination in the light at a temperature of 27±3° C. Germination plates were examined weekly, over a period of about four weeks, and germinating embryos were transferred to MAGENTA®) boxes containing 50-100 ml of MSG₃ medium for conversion to plantlets. MAGENTA® boxes containing developing plantlets were incubated in the light at 27±3° C. for about eight to twelve weeks until they could be transferred to soil or potting mix in a greenhouse or outdoor conditions under shade. Despite the differences in the number of embryos developed to a harvestable quality on the different types of membranes, an experiment using three embryogenic lines demonstrated that the percentage of those embryos that could be germinated was not significantly different. This suggests that a switch in the type of membrane used during embryo development, while it affects embryo production, does not have long-term effects on the viability of the embryos.

As seen from the data shown in Tables 9, 11, and 12, the characteristic of the different membrane treatments that had the largest effect on proliferative growth of embryogenic cells was the degree to which the support membrane itself absorbed or reacted with liquid from the media below it, resulting in swelling of the fibers making up the membrane. Membranes made of less absorbent and less acid-reactive materials (those made of polyester and ETFE) generally promoted better growth, perhaps because in failing to absorb as much liquid, they allow more of the liquid and the large molecules contained in it, such as plant growth regulators, to pass through the membrane and enter the plant tissue. This characteristic of the polyester and ETFE membranes also had a strongly significant promotive effect on regeneration of high quality embryos from the embryogenic cell lines when the cultures were both maintained/proliferated and embryos developed/matured on the same type of membrane sequentially.

It had been expected that mesh size, and factors affecting it, might have a significant effect on growth or development. However, the data show that there were no significant correlations between either the number of fibers per cm of the membrane or the dry mesh opening size with either growth or embryo development. The values supplied by the manufacturer for mesh size and percent open area of the membrane when dry were considered independently of the capacity of the fibers to absorb liquid and swell (particularly in the nylon membranes, this would decrease both the effective mesh size and percent open area measurements). There was also no correlation between the percent open area and growth, while with embryo development there was only a weak correlation (R<0.35) with the percentage open area.

Similarly, other characteristics, such as the thickness of the membranes or the dry diameter of the individual fibers making up the membranes, did not appear to have any significant effect either on growth of the cultures or development of harvestable embryos when considered independently of the fiber type. Thread diameter had a significant effect only when considered within fiber type; both proliferative growth and embryo development/maturation were best on polyester or ETFE membranes with greater than 40 micron fiber diameter, but because the ETFE membranes were, at the time this experiment was done, only available in the largest fiber diameter class, an apparent promotive effect for the larger fiber diameter class is likely to be confounded with fabric type and absorbance.

Finally, we found that the use of the membrane supports greatly facilitated the transfer of plant cell material between different media and culture phases. While the use of nylon supports had previously been claimed to facilitate the transfer of plant material, we found that removal of embryos at the harvest stage and germination stage described above was easier when supports made of non-swelling fibers (e.g. polyester) were used. The use of any of these types of support membranes did not have any long-term adverse effects on germinability of the embryos harvested.

Thus, any of the fiber types could be used in supports to grow and develop embryos, but the main significant effect is one of absorbance characteristics of the type of fiber used in the membrane supports, namely that non-absorbent, non-acid-reactive fibers in liquid-permeable membrane supports (polyester or ETFE, in this example) resulted in best proliferation, best embryo differentiation, and easiest transfer.

### EXAMPLE 5

This teaches a further improvement over the traditional method of culture directly on a gelled medium, by spreading pine embryogenic tissues on polyester support membranes laid over a biphasic system incorporating a thin layer of liquid medium, which may be absorbed into a filter paper, over a gelled medium. This was demonstrated to be non-detrimental to the growth and subsequent development of high quality somatic embryos from pine embryogenic tissues.

In this example the methods described above for the Southern yellow pine P. taeda are extended to another pine species, Pinus radiata, which is known to those skilled in the art of dendrology to be divergent from the preceding species, as demonstrated by the fact that even when it is grown sympatrically it does not readily hybridize with the preceding species. Furthermore, in the present example, the maintenance media described in U.S. Pat. No. 5,565,355 (which is hereby incorporated by reference) as being sufficient to promote growth of P. radiata are used as basal media in our improved. These examples serve to illustrate that any nutrient media that have been established as sufficient to promote growth or embryogenesis of the target tissue of any species may be employed in conjunction with the present method without undue experimentation.

In this example, immature seed cones were collected from several P. radiata sources located in breeding orchards in New Zealand. The media and methods used to handle the cultures once they were initiated are those disclosed in U.S. Pat. No. 5,565,355. Once cultures were extruded, cryopreserved, and then retrieved, they were subjected to the procedures described in this example.

To test the growth of Pinus radiata cells on support membranes over gelled medium as described in Examples 3-4 above or biphasic media, cells of lines derived from five different P. radiata families were plated, as described in Example 4 above, on the following treatments:

A. Cells were placed directly onto maintenance medium with no support filter or membrane.

B. Cells were placed on a nylon support membrane (NITEX with pre-wetting pore size 35, commercially available from SEFAR), and this membrane bearing the embryogenic cells was then placed on maintenance medium.

C. Cells were placed on a polyester support membrane (PECAP with pre-wetting pore size 35, commercially available from SEFAR), and this membrane bearing the embryogenic cells was then placed on maintenance medium.

D. Cells were placed on a filter paper (WHATMAN No. 3), which had been wetted with a few drops of liquid maintenance medium (the same as the gelled maintenance medium but lacking gelling agent), and this filter paper bearing the embryogenic cells was then placed on maintenance medium.

E. Cells were placed on a polyester support membrane (PECAP with pre-wetting pore size 35, commercially available from SEFAR), and this membrane bearing the embryogenic cells was then placed over gelled maintenance medium on which had been placed a filter paper (WHATMAN No. 3) that had been wetted with a few drops of liquid maintenance medium (the same as the gelled maintenance medium but lacking gelling agent).

F. Cells were placed on a polyester support membrane (PECAP with pre-wetting pore size 35, commercially available from SEFAR), and this membrane bearing the embryogenic cells was then placed over gelled maintenance medium on which had been placed a filter paper (WHATMAN No. 3) that had been wetted with a full milliliter of liquid maintenance medium (the same as the gelled maintenance medium but lacking gelling agent).

The cells on their supports were weighed (cells in treatment A were weighed on a polyester support membrane and then gently scraped off the membrane using sterile forceps, or a sterile plastic one-use cell harvester, onto the gelled media) and were then maintained on the same supports, with weighing and transfer to fresh gelled medium biweekly for four transfer cycles.

TABLE 13
Growth Of Pinus radiata Cell Cultures During
6 Weeks On Various Support and Biphasic Treatments

| P. radiata | Average (n = 3) difference between weights (in grams) | | | | | |
|---|---|---|---|---|---|---|
| Embryogenic | at start and end of 6 weeks | | | | | |
| Cell Line | Treatment A | Treatment B | Treatment C | Treatment D | Treatment E | Treatment F |
| Family F | 1.02 +/− 0.08 | 1.85 +/− 0.38 | 2.77 +/− 0.58 | 2.43 +/− 0.30 | 3.46 +/− 1.72 | 2.34 +/− 0.12 |
| Family X | 1.47 +/− 0.31 | 2.28 +/− 0.14 | 3.48 +/− 0.50 | 3.90 +/− 0.25 | 4.35 +/− 0.74 | 3.08 +/− 0.68 |
| Family K | 1.21 +/− 0.07 | 2.00 +/− 0.70 | 3.09 +/− 0.36 | 2.31 +/− 0.26 | 1.53 +/− 0.65 | 1.22 +/− 0.78 |
| Family D | 1.35 +/− 0.03 | 3.31 +/− 0.33 | 3.70 +/− 0.60 | 2.31 +/− 0.57 | 2.52 +/− 1.38 | 1.68 +/− 0.48 |
| Family Q | 0.40 +/− 0.04 | 1.00 +/− 0.23 | 1.75 +/− 0.30 | 1.01 +/− 0.22 | 0.87 +/− 0.32 | 0.50 +/− 0.34 |

As can be seen in Table 13, for no cell line was the average growth over a period of six weeks less for cells grown over support membranes and biphasic treatments than for cells grown directly on gelled medium. Because the membrane supports facilitate rapid transfer and weighing with minimal manipulation of the cells, damage that cells sustain during transfer between gelled media without membrane supports, as described in Example 3 above, may account for some of the difference between Treatment A and the other treatments. Also, for all P. radiata cell lines, as had been shown for P. taeda and P. rigida hybrids in Example 4 above, growth on a polyester membrane support was superior to growth on a nylon membrane support. It was also superior to growth on a filter paper support alone, a technique which has been commonly used for culture of P. radiata cells and embryogenic cells of some other woody plants. The same patterns were observed in data analyzed for a single two-week transfer period. Embryos were subsequently successfully developed, matured, and germinated from P. radiata cells of these lines that had been maintained on polyester support membranes. These data suggested that polyester membrane supports and biphasic media could be used to facilitate washing, decontamination, and selection applications as described in the examples below. This also illustrates that the present invention is not limited to any single basal culture nutrient medium formulation. It should be understood that any nutrient media commonly used in Pinus somatic embryogenesis will be suitable for use with this method.

### EXAMPLE 6

This example teaches a method for improving decontamination of plant tissue (i.e. the eradication of microbial contaminants), in this example from pine embryogenic tissues. In particular, this example teaches an improved washing procedure whereby the use of support membranes, preferably support membranes made of a non-swelling material such as polyester or a fluoropolymer instead of nylon support membranes, facilitates the rapid removal of the plant cells from a bacterial contaminant, without concomitant loss of valuable pine material through adherence to the membrane support.

The procedures for culture and subculture of embryogenic cells of pine described in Examples 1-2 above were used for all growth phases in the following examples, with the results as described in the following examples.

Loblolly pine cell lines were used which had been grown and maintained in liquid suspension cultures as described in Example 4 prior to and after cryopreservation. One to five milliliters of embryogenic suspension was pipetted onto a nylon or polyester membrane support of the same types as used in Example 2 above and the support bearing the embryogenic tissue was placed on gelled medium by the method described in Example 2 above. Agrobacterium tumefaciens strain EHA105 was dripped over the plant material and co-cultivated in the dark at 23±2° C. for approximately 48-72 hours, by which time significant overgrowth of the bacteria on the plant cells was evident. Those skilled in the art of plant pathology recognize that the present method would apply to many different bacterial strains, media, and infection protocols.

Following the observation of significant bacterial overgrowth the eradication of bacteria from the cultures was carried out as follows. The contaminated cells were re-suspended into fresh DCR₃ liquid wash medium (Table II) containing eradicants such as 200-400 mg/L TIMENTIN®, 250-500 mg/L carbenicillin, or 250-500 mg/L cefotaxime. Resuspension was initiated by grasping the membrane support bearing the infected cells, using forceps, and rolling or folding it so that it could be taken up and placed into the liquid. The liquid was then agitated to get the cells into suspension, and the membrane support was scraped with sterile forceps if cells appeared to be adhering to it. Once the cells were in suspension, the membrane was removed with sterile forceps.

The contaminated cells were cultured in the wash medium briefly, then plated onto fresh support membranes again by placing the fresh sterile support membranes in a sterile Buchner funnel, pipetting the suspension of plant cells onto the membranes, and again suctioning the liquid medium from the tissues using a mild vacuum. In this example, both the nylon and polyester membranes used had a dry mesh size of 35 microns. The bacterial cells, ranging in size from 5 microns in diameter, are largely washed through the mesh of the fabric membrane support, while the much larger plant cells are retained. Thus, the effective mesh size desirable for use in this method would depend on the size of the microbial cells to be eliminated, as well as the size of the contaminated plant cells being cultured. The plant cells remaining on the membranes were then resuspended in fresh sterile liquid wash medium and the amount of plant tissue thus transferred was measured by settled cell volume, while the presence of the bacteria in the collected post-rinse medium was observed both by its cloudy appearance and by counting colonies that arose from culturing it on a rich bacterial medium. For each successive wash cycle, the cells were again resuspended in and briefly cultured in fresh sterile wash medium by agitating the membrane bearing the cells in the liquid, again removing cells that appeared to be adhering by gently scraping with forceps. The cells were then re-plated on fresh membrane supports over Buchner funnels and rinsed with fresh sterile medium. This procedure was repeated until the colony counts of the collected post-rinse medium revealed that the bacteria were no longer present in sufficient inoculum to re-grow even in rich medium or until the plant cells had become inviable (measured by lack of subsequent growth, presumably due to successive damage).

The results were that in washing procedures using either nylon or polyester support membranes for transferring the cells between the co-cultivation, wash, and post-wash culture media, bacterial colony counts from the cultured post-rinse medium showed that the number of colonies arising from bacterial inoculum remaining after the washes decreased significantly after washing, allowing the growth of the remaining viable plant cells to proceed without the very rapid bacterial overgrowth observed in controls not washed with eradicant-containing medium.

We further observed that the plant cells to be decontaminated were released very easily from the polyester support membranes, requiring little scraping, while the nylon membranes retained many cells (both bacterial and plant cells), and more scraping was usually required for resuspension. Thus, it seemed that more washes could be performed in an equivalent period of time when polyester supports were used, and the final amount of viable pine cells in culture after multiple washes remained closer to the initial amount.

Furthermore, because the plant cells were released more easily from the polyester support membranes, while the nylon membranes retained many cells, it seemed that more washes could be performed in an equivalent period of time when polyester supports were used, and the final amount of viable pine cells in culture after multiple washes remained closer to the initial amount.

This experiment was repeated subsequently using nylon, polyester, and fluoropolymer (ETFE) support membranes of the same and larger (85 micron) mesh sizes, and in some treatments changing from one membrane type in the beginning of the experiment to another for subsequent washes. The results were similar in that more viable pine cells were recovered following decontamination in the treatments in which polyester or fluoropolymer support membranes were used, and the best recovery resulted from a treatment in which fluoropolymer support membranes were used for the washing step.

The improved decontamination procedure experiment was repeated also subsequently tested using polyester support membranes for transferring the cells between the co-cultivation, wash, and post-wash culture media, in an experiment in which the infections consisted either of both a less virulent strain of the bacterium (GV2260), Agrobacterium with no virulence-enhancing plasmid, and the highly virulent strain EHA105 with the virulence-enhancing plasmid pTOK47 (Wenck et al. 1999), were used. The improved washing method described in this example was capable of eradicating either bacterial strain, even the virulence-enhanced inoculum, without excessive damage to the plant cells, as measured by their ability to resume growth following completion of the bacterial decontamination eradication procedure.

### EXAMPLE 7

This example teaches another improved method for improving the eradication of bacteria from plant tissues. Those skilled in the art of plant tissue culture will recognize that this improved decontamination method is applicable to any tissues of any plant species, such that the tissues are able to be spread over the surface of a support membrane. In particular, this method is applicable to callus cultures, suspension cultures, or embryogenic cultures. This example demonstrates the use of the method with pine embryogenic tissues. In particular, this example teaches an improvement over the traditional method of application of antibiotics in the gelled medium, by spreading pine embryogenic tissues on polyester support membranes laid over a biphasic system incorporating antibiotics in a liquid medium, which was demonstrated to be non-detrimental to the growth and subsequent development of high quality somatic embryos from pine embryogenic tissues. This method may be used together with the improved method described in Example 6 or independently of it.

Loblolly pine cell lines were used which had been grown and maintained as described in Examples 3-4 above, and purposely infected with bacteria as described in Example 6 above. Support membranes bearing plant tissue were then placed on gelled DCR₂ maintenance media with various decontamination treatments. These treatments consisted of antibiotics (cefotaxime, carbenicillin, AUGMENTIN® or TIMENTIN®) as eradicants incorporated into the gelled DCR₂ maintenance or selection media, the antibiotics incorporated into liquid DCR₃ pipetted in a thin film over gelled DCR₂ maintenance media lacking antibiotics, or the antibiotics incorporated into liquid DCR₃ which was saturated into a filter paper laid on gelled DCR₂ maintenance media lacking antibiotics. Support membranes bearing control cells were placed either on gelled DCR₂ maintenance media, over liquid DCR₃ pipetted in a thin film over gelled DCR₂ maintenance media lacking antibiotics, or over a filter paper saturated with liquid DCR₃ and laid on gelled DCR₂ maintenance media lacking antibiotics. Decontamination and maintenance were continued for a period of approximately 12 weeks with transfer of the polyester support membranes, bearing the cells, every 14-21 days.

Results showed that the maintenance and proliferative culture of cells over a bi-layer formed by liquid DCR₃, in some treatments containing the eradicant antibiotics, pipetted in a thin film (1-3 ml, usually 1.5 ml) over gelled DCR₂ maintenance media, or saturated into a filter paper laid on gelled DCR₂ maintenance media, was not detrimental (and for some cell lines even appeared to be beneficial) to the growth of embryogenic cells, either of loblolly pine (4 lines from two unrelated families, designated with “P”) or hybrid pine (lines designated “H”) as seen in Table 14 below.

TABLE 14
Growth Of Pine Embryogenic Cells On Polyester Membrane Supports
Over Biphasic Culture Media (Gelled Phase Under Liquid Phase)

| | Average growth over a one-month period (two transfers) | | | |
|---|---|---|---|---|
| Cell | No liquid | Liquid | Liquid phase | Liquid phase with |
| Line¹ | phase² | phase³ | with cefotaxime⁴ | TIMENTIN ®⁵ |
| P1 | 5.53 + 0.98 | 7.24 + 0.20 | 7.20 + 0.28 | 7.71 + 0.42 |
| P2 | 2.03 + 0.17 | 2.37 + n.d. | 1.93 + 0.24 | 2.58 + 0.09 |
| P3 | 5.27 + 2.62 | 9.36 + 0.25 | 6.75 + 0.25 | 9.00 + 0.56 |
| P4 | 2.84 + 0.40 | 11.18 + 0.34 | 8.97 + 0.19 | 10.79 + 0.90 |
| H1 | 4.53 + 0.73 | 5.82 + 0.29 | 4.53 + 0.28 | 5.73 + 0.52 |
| H2 | 5.43 + 0.59 | 11.34 + 0.66 | 8.76 + 0.63 | 11.58 + 0.67 |

¹P1 = P. taeda cell line 1, P2 = P. taeda cell line 2, P3 = P. taeda cell line 3, P4 = P. taeda cell line 4, H1 = Hybrid pine cell line 1, H2 = Hybrid pine cell line 2.
²Non-biphasic.
³Liquid phase same medium as gelled phase except without gelling agent.
⁴Liquid phase containing 500 mg/L of cefotaxime.
⁵Liquid phase containing 400 mg/L of TIMENTIN ®.

Comparison with the non-biphasic control shows that the biphasic method was also not detrimental (and for some cell lines even appeared to be beneficial) to the embryogenicity of the cultures, as the results showed when pine cell cultures maintained in the treatments described above were subsequently transferred to embryo development medium MSG₁ as described in Example 4, and embryos were harvested after 12 weeks and carried through germination as described in Example 4 (Table 15).

TABLE 15
Embryogenicity Of Cultures Submitted To Biphasic Maintenance
Treatments Prior To The Onset Of Embryo Development

| | Average Number Of Embryos Harvested Per Plate (% germination) | | | |
|---|---|---|---|---|
| | | | Liquid | Liquid phase |
| Cell | No liquid | Liquid | phase with | with 400 mg/L |
| Line¹ | phase² | phase³ | cefotaxime⁴ | TIMENTIN ®⁵ |
| P1 | 45.7 ± 31.4 | 39.7 ± 12.1 | 87.0 ± 37.5 | 101.0 ± 41.6 |
| | (40%) | (15%) | (10%) | (64%) |
| P2 | 110.0 ± 29.6 | 94.3 ± 26.1 | 81.7 ± 44.5 | 85.5 ± 3.5 |
| | (60% | (35%) | (66%) | (80%) |
| P3 | 33.0 ± 5.6 | 12.0 ± 10.4 | 14.3 ± 4.2 | 58.7 ± 14.4 |
| | (13%) | (11%) | (21%) | (65%) |
| P4 | 9.3 ± 2.5 | 76.3 ± 36.1 | 34.0 ± 16.1 | 56.7 ± 15.0 |
| | (13%) | (28%) | (36%) | (30%) |
| H1 | 46.3 ± 18.2 | 122.7 ± 26.6 | 152.0 ± 40.8 | 151.5 ± 21.9 |
| | (50%) | (87%) | (96%) | (88%) |
| H2 | 73.3 ± 7.23 | 119.3 ± 76.9 | 77.0 ± 10.4 | 118.0 ± 27.2 |
| | (93%) | (93%) | (88%) | (85%) |

¹P1 = P. taeda cell line 1, P2 = P. taeda cell line 2, P3 = P. taeda cell line 3, P4 = P. taeda cell line 4, H1 = Hybrid pine cell line 1, H2 = Hybrid pine cell line 2.
²Non-biphasic.
³Liquid phase same medium as gelled phase except without gelling agent.
⁴Liquid phase containing 500 mg/L of cefotaxime.
⁵Liquid phase containing 400 mg/L of TIMENTIN ®.

Furthermore, in cells contaminated with Agrobacterium, eradicants presented to the cells in liquid DCR₃ pipetted in a thin film over gelled DCR₂ maintenance media, or saturated into a filter paper laid on gelled DCR₂ maintenance media were as or more successful in suppressing the growth of Agrobacterium than eradicants incorporated in the gelled DCR₂ media, with the overall use of only 7.5% of the amount of eradicant per plate in which it was applied (1.5 ml liquid vs. 20 ml gelled medium). Moreover, the flexibility of the bi-layer liquid eradicant surface application method allowed even more savings in eradicant. Unlike gelled media (which must be made fresh some days before needed and in which the eradicants have a short half-life), aliquots of liquid eradicant-containing media can be frozen almost indefinitely for use when required. The cultures that would still suffer microbial regrowth were readily distinguishable from those that had already undergone sufficient eradication, after a transfer onto fresh gelled medium lacking incorporated eradicant, whereas with eradicant incorporated in the medium these were not distinguishable. When, as with the improved method, it could be rapidly determined which cultures are no longer contaminated, the eradicant that would have been used for them could be left out of the culture simply by not adding the liquid phase over the gelled maintenance medium, while a liquid eradicant overlay can be added without significant delay to those cultures requiring it.

Agrobacterium contamination has been reported as recurring sometimes after long periods of time. With many species, eradicants are incorporated in all culture media used after the initial infection, including selection media, proliferation media, media to induce the formation of organs or the development of somatic embryos, media to elongate or mature organs or embryos that are formed, and regeneration media. For pine embryogenic cells, incorporation of eradicants into the embryo development and maturation media has been difficult due to the high temperature of polymerization of the media resulting from the incorporation of a high level of polyethylene glycol. Therefore, loblolly and hybrid pine cell lines grown and maintained as described in Examples 3-4 above were placed on polyester support membranes over gelled MSG₁ embryo development and maturation media as described in Example 4 above, except that some of the development and maturation media were overlaid with various eradication treatments under the polyester support membranes. The treatments consisted of either no liquid phase, or a liquid phase identical to the gelled phase (except that gelling agent and activated charcoal were omitted) and incorporating either cefotaxime or TIMENTIN® as an eradicant. Three replicate plates were generated from each of six embryogenic cell lines (two from each of two P. taeda families and one hybrid pine family) for each of the treatments, and assessed for the ability of the cultures to develop high quality harvestable stage 3 embryos. Dishes were incubated in a dark growth chamber at 23±2° C. The membrane supports were transferred to new petri dishes containing fresh medium every 3 weeks. At week 9, stage 3 embryos were counted and those deemed suitable for germination were harvested. The results are shown in Table 16 below.

TABLE 16
Effect Of Placing Cells On Polyester Support Membranes Over
Eradicants In Liquid Phase of Biphasic Embryo Development
and Maturation Media

| | Number of Harvestable Cotyledonary Stage Embryos | | |
|---|---|---|---|
| | Developed Over | | |
| | | Liquid phase with | Liquid phase with 400 |
| Cell Line¹ | No liquid phase² | cefotaxime³ | mg/L TIMENTIN ®⁴ |
| P1 | 36.0 ± 7.6 | 208.0 ± 94.8 | 226.0 ± 67.9 |
| P2 | 94.0 ± 38.9 | 269.0 ± 23.3 | 217.0 ± 62.9 |
| P3 | 28.0 ± 17.7 | 25.0 ± 17.7 | 26.0 ± 7.8 |
| P4 | 103.0 ± 49.5 | 18.0 ± 1.0 | 16.0 ± 7.0 |
| H1 | 173.0 ± 31.8 | 200.0 ± 51.2 | 233.0 ± 10.1 |
| H2 | 166.0 ± 67.1 | 210.0 ± 67.1 | 300.0 ± 24.9 |

¹P1 = P. taeda cell line 1, P2 = P. taeda cell line 2, P3 = P. taeda cell line 3, P4 = P. taeda cell line 4, H1 = Hybrid pine cell line 1, H2 = Hybrid pine cell line 2.
²Non-biphasic (gelled phase only).
³Liquid phase containing 500 mg/L of cefotaxime.
⁴Liquid phase containing 400 mg/L of TIMENTIN ®.

As shown in Table 16, the application of eradicants in a liquid phase between the gelled medium and the cells borne on a polyester support membrane does not result in detriment to embryo development in five of the six embryogenic lines (in fact, it appears to be beneficial in most of the lines), and embryos could be developed from all lines tested.

Embryos developed during biphasic application of eradicants as described in the paragraph above were subjected to a germination test to determine whether the biphasic application of eradicants under polyester support membranes over gelled MSG₁ embryo development and maturation media had affected their germinability. The treatments during embryo development consisted of either no liquid phase, or a liquid phase identical to the gelled phase (except that gelling agent and activated charcoal were omitted), or the same liquid phase incorporating either 200 or 400 mg/L TIMENTIN® as an eradicant. Germination was carried out as described in Example 4, and the results are shown in Table 17 below.

TABLE 17
Effect Of Subsequent Germination Of Placing Cells On Polyester
Support Membranes Over Eradicants In Liquid Phase of Biphasic
Embryo Development And Maturation Media

| | Percentage Of Embryos Germinating After Development On | | | |
|---|---|---|---|---|
| | Polyester Support Membranes | | | |
| | | | Liquid phase | Liquid phase |
| | | | with 200 mg/L | with 400 mg/L |
| Cell | No liquid | Liquid | TIMENTIN ® | TIMENTIN ® |
| Line¹ | phase² | phase³ | Over Gelled Phase | Over Gelled Phase |
| H1 | 92.5% | 95.0% | 72.5% | 95.0% |
| P1 | 42.5% | 55.0% | 35.0% | 17.5% |
| P2 | 62.5% | 67.5% | 52.5% | 47.5% |

¹P1 = P. taeda cell line 1, P2 = P. taeda cell line 2, H1 = Hybrid pine cell line 1.
²Non-biphasic (gelled phase only, no liquid phase).
³Liquid phase without eradicants over gelled phase.

As shown in Table 17, the application of a liquid phase between the gelled medium and the cells borne on a polyester support membrane, whether or not it contains TIMENTIN®, does not result in a significant detriment to embryo germination, and embryos could be germinated from all lines tested. Thus, if required due to long-term resurgence of bacterial growth in a culture, the method described in this example allows the application of eradicant even in the presence of embryo development and maturation media in which it could not otherwise be effectively incorporated.

### EXAMPLE 8

This example teaches a combination of the biphasic methods used in Examples 5 and 7 above and the washing method taught in Example 6 above, which is here applied to organogenic tissue rather than to embryogenic tissue, for improving the decontamination of plant cultures by eradication of bacterial contaminants. In particular, this example teaches a method of spreading callus tissues or suspension cell cultures derived from callus tissues from species comprising both herbaceous and woody plants, onto support membranes for a washing procedure that facilitates the rapid removal of the plant cells from a bacterial contaminant, without concomitant loss of valuable plant material through adherence to the membrane support. Furthermore, this example teaches an improvement over the traditional method of application of eradicants in the gelled medium, by spreading plant tissue on polyester support membranes laid over a biphasic system incorporating antibiotics in a liquid medium over the gelled medium, which allows added flexibility in the administration of eradicants and was demonstrated to be non-detrimental to the growth of the plant tissue.

Tobacco suspension cells were prepared as described in Example 1, and equal weights were plated either directly on medium for treatment A or on polyester support membranes for treatments B-F. For the purposes of this example, the tobacco cells were then purposely infected with a bacterial contaminant sensitive to cefotaxime, as described for Agrobacterium co-cultivation of pine tissue in Example 6 above. After 48 hours, it was determined that all plates of the tobacco cells were covered with a lawn of bacteria.

For Treatment A, infected cells were then transferred to gelled MS growth medium as described in Example 1 above, except that it contained an antibiotic at levels sufficient to kill the bacterial contaminant (this control procedure is the traditional method of eradication to which the improved method is being compared), namely 500 mg/L cefotaxime. Those skilled in the art of plant tissue culture will recognize that other antibiotics can be used as eradicants in the same method.

For the remaining treatments, infected cells were washed with liquid MS medium (the same as gelled MS medium in Example 1, but lacking gelling agent), using a washing procedure similar to the procedure described for pine embryogenic cells in Example 7 above. The cells on their polyester support membranes were first rinsed with 20 ml of liquid MS medium over a vacuum manifold, drawing off much of the visible bacterial contamination. It was then simple to release the plant cells from the polyester support membranes by dipping the membranes into sterile liquid medium containing antibiotic (more scraping had been necessary when nylon membranes were tried). Cells that adhered to each other in large clumps in the liquid medium were gently broken apart with a large-bore pipette. The cells in the liquid wash medium were agitated 2-4 hours at a speed of 134 rpm in the antibiotic-containing wash medium, and then re-plated on polyester support membranes as described in Example 7 rinsed with fresh liquid medium, and the procedure was repeated. Following the second wash, cells were re-plated on fresh membrane supports, and placed over medium to be maintained as follows:

Treatment B: the polyester support membrane was placed on MS gelled medium lacking cefotaxime.

Treatment C: the polyester support membrane was placed on MS gelled medium lacking cefotaxime, and over a 1.5 ml liquid phase comprising the same medium without gelling agent.

Treatment D: the polyester support membrane was placed on MS gelled medium lacking cefotaxime, and over a filter paper soaked with 1.5 ml liquid medium (the same medium without gelling agent).

Treatment E: the polyester support membrane was placed on MS gelled medium lacking cefotaxime, and over a 1.5 ml liquid phase comprising MS medium without gelling agent but containing 500 mg/L cefotaxime.

Treatment F: the polyester support membrane was placed on MS gelled medium lacking cefotaxime, and over a filter paper soaked with 1.5 ml liquid medium containing 500 mg/L cefotaxime.

The cells were incubated in the lighted growth chamber set at 27±3° C. and were observed for any bacterial regrowth. Small amounts of tissue were also periodically removed onto a rich bacterial medium to test for bacterial regrowth from contamination that was not readily visible in the plant medium. Cells were transferred to fresh medium every three weeks whether contamination was observed or not.

Treatments B, C, and D, all treatments lacking any eradicants, were heavily contaminated with bacterial regrowth within two days. Treatment E was somewhat contaminated with bacterial regrowth after five days, and Treatment F was somewhat contaminated with re-growth after ten days. As soon as bacterial regrowth was noticed on any of these support membrane treatments, the cells were again washed using fresh polyester support membranes as described above, and the membranes bearing the cells were then plated on fresh gelled and liquid media according to the treatments described. Bacterial regrowth was not seen again on these treatments, over a period of maintenance of greater than three months.

After two weeks, the only plates showing visible bacterial regrowth were in treatment A, which contained the eradication agent cefotaxime in the medium but on which polyester support membranes were not used. Avoiding the contaminant as much as possible, the plant cells were transferred onto fresh plates containing eradicant in the gelled medium as soon as the contaminant regrowth was noticed. Only after two more transfers onto eradicant-containing medium, over a total of nine weeks, was there no visible bacterial regrowth on Treatment A.

In summary, the traditional treatment without benefit of either the washing treatment facilitated by polyester support membranes, or the eradicants administered in the liquid under the support membranes in the biphasic application, allowed bacteria to re-grow several times up to nine weeks after the initial infection. In contrast, the washing treatments, facilitated by the polyester support membranes, were sufficient to remove all bacterial contamination, because no bacterial regrowth was seen after the second wash procedure, even on plates that contained no eradicants whatsoever (treatments B-D). In addition, the application of eradicant in the biphasic system, in liquid medium under the polyester support membrane (treatments E and F), was sufficient to delay the need for a second wash treatment. In the improved method, the much shorter exposure to the eradicant may be important for certain types of cells, as several commonly used eradicants have been reported to have effects on regeneration of plant cells.

These results have since been repeated using the alternative eradicants AUGMENTIN® and TIMENTIN®. Those skilled in the art of plant tissue culture will recognize that the improved method can be used with a variety of eradicants, including those mentioned as well as miconazole, rifampicin, and other microbial control agents that can be incorporated into a liquid medium.

The improved method was also employed to control Agrobacterium contamination of petiole explants in a cottonwood (Populus sp.) culture. The petiole explants, though rather different in form from the callus and suspension cells used above, were able to be washed easily using polyester support membranes and cultured over similar media and membrane treatments as described for the tobacco above. Best survival of plant material (as evidenced by growth of callus on the petiole explants) was on polyester support membranes over biphasic media; this experiment also demonstrated that use of the polyester support membranes over biphasic media containing both a selection agent and an eradicant antibiotic in the liquid phase was not detrimental to subsequent regeneration of shoots from the callus.

Though the invention described here has been tested particularly in transformation of pine somatic embryogenic cultures and tobacco suspension cultures by Agrobacterium, a eubacterium, it can also be used for eradication with Agrobacterium transformation of many types of small explants or callus of any species that can be shown to grow on polyester or fluoropolymer membranes over gelled media. The method can also be applied to eradication of other types of recurring bacterial contaminants from valuable cultures.

### EXAMPLE 9

This example teaches the use of the improved method, taught above for decontaminating embryogenic and organogenic callus of bacteria, for obtaining the eradication of fungal contaminants from small explants. This serves to show that this method would be applicable to any type of culture, such as a callus or nodular culture, or even small explants such as pieces of petiole, leaf, or root, of a variety of species. In addition, this method is applicable to the eradication of a variety of microbial contaminants. In particular, this example teaches a method of spreading callus tissues, nodule culture tissues, suspension cell cultures derived from callus tissues, or even explants such as pieces of petiole, from a variety of species comprising both herbaceous and woody plants, onto support membranes for a washing procedure that facilitates the rapid removal of the plant cells from a fungal contaminant, without concomitant loss of valuable plant material through adherence to the membrane support. As in the previous example, this is followed by application of the further improvement over the traditional method of application of eradicants in the gelled medium, which comprises spreading plant tissue on polyester support membranes laid over a biphasic system incorporating eradicants in a liquid medium over the gelled medium, which allows added flexibility in the administration of eradicants.

Eucalyptus camaldulensis leaves were explanted from seedlings for the production of callus, and then discovered to be contaminated with an unknown fungus. Contaminated explants were shaken together to obtain the same degree of contamination for all explants to be used in this decontamination example. Contamination of each explant with the fungus was verified by streaking the explant on a potato dextrose agar plate, and observing independent growth of the fungus on the potato dextrose agar.

Approximately half the Eucalyptus leaf explants were then treated to decontamination by washing and collection of the tissue using polyester support membranes after each wash, similar to the improved decontamination method described in Example 6, split among five treatments:

1. no fungicide or fungistatic compound present in the wash medium

2. Benomyl added to the wash medium at 100 mg/L

3. Miconazole added to the wash medium at 50 mg/L

4. Nystatin added to the wash medium at 100 mg/L

5. Thiophanate added to the wash medium at 100 mg/L

Those skilled in the art of plant tissue culture will recognize that other water-soluble or water-miscible antifungal agents could be used as eradicants in the same method.

Following the washing of half the material according to the protocol described in Example 5 in the treatments described in the paragraph above, using three washes of 30 minutes each, the washed and non-washed material was then divided over the same support membrane treatments A-F as described in Example 8, except that nested within each support membrane treatment was the prior washing or lack of washing treatment, and within each of these latter treatments, the five fungicidal treatments listed above were further nested, wherein the antifungal agents benomyl, miconazole, nystatin or thiophanate were added to the medium subtending the tissue and supports in place of cefotaxime. Finally, non-contaminated explants were submitted to all treatments to determine the effects of the treatments on plant regeneration in the absence of fungus. Five explants were plated for each treatment combination.

The explants were then cultured in a growth chamber set at 27±3° C. and under low light, and were transferred biweekly using the same support membranes but supplying fresh sterile liquid and solid medium. At each transfer all explants were observed for any fungal regrowth (upon which explants were recorded as contaminated), as well as for the formation of callus and organogenic formation of shoots.

It was found that the four antifungal agents were not all equally effective in either decontamination or lack of detriment to the plants. The benomyl treatments, both in the washing and incorporated into the medium below the polester supports, was largely ineffective in decontamination and also elicited considerable phenolic release into the medium even from the successfully decontaminated plant tissue. Most successful were the miconazole treatments, in which decontamination succeeded in all but 7.5% of the contaminated explants, a decontamination rate of 92.5%.

The polyester support membranes were shown to be quite suitable for collecting and transferring small leaf explants for the same reasons they were suited to collection and transfer of embryogenic and callus cells in the improved methods described in Examples 6 and 8.

The membrane support treatments in the absence of washing were sufficient to accomplish the best decontamination, and of these the best decontamination rate was seem in the treatment wherein the polyester support membrane was laid over a biphasic eradication medium in which the antifungal agent was incorporated into both the gelled medium and a liquid phase in a filter paper between the gelled medium and the support membrane bearing the plant tissue. This lack of benefit from the washing treatments shown in prior examples to be useful for the eradication of bacteria, may be due to encouragement of fungal cell growth by the increased wetting of the tissue relative to the control (no wash, no membrane support treatment, explants placed directly on the gelled medium). The membrane support treatments, in contrast, tend to present a slightly drier plant tissue culture than the control because the cells are not in direct contact with the gelled medium.

In the absence of fungus and antifungal agents, shoot formation occurred at approximately the same pace on all support membrane treatments, indicating that neither culture over membrane supports nor over membrane supports on biphasic media were deleterious to organogenic development. However, the wash treatments delayed shoot formation by about two weeks on most explants, and, as noted above, were not shown to be of added benefit for fungal decontamination in this case. In the decontaminated explants, the best shoot formation was also seen to occur in the biphasic treatments, wherein the polyester support membrane was laid over a biphasic eradication medium in which the antifungal agent was incorporated into both the gelled medium and a liquid phase in a filter paper between the gelled medium and the support membrane bearing the plant tissue. Thus, the best improved method for decontamination of fungus from explants incorporates the treatment on biphasic media.

This example further demonstrates the improved decontamination treatment using biphasic media is suited for use with many types of small explants or callus of any species that can be shown to grow on polyester or fluoropolymer membranes over gelled media, and that the method can be successfully applied to eradication of other types of recurring microbial contaminants from valuable cultures.

### EXAMPLE 10

This example teaches the use of the improved methods for obtaining the eradication of a different type of contaminant from plant tissue, in this case a mycoplasma. This experiment demonstrated that the method is applicable to ridding plant cell cultures of a variety of microbial contaminants.

A Pinus taeda culture and two P. radiata embryogenic cultures grown on the respective maintenance media described in Examples 4 and 5 were discovered to be contaminated with Mycobacterium gordonae, which caused the plant cells to exhibit an orange tinge and subsequently mycobacterial overgrowth. The mycobacterium was then used to inoculate an additional culture of P. taeda for experimental purposes. Contamination of each culture could be more readily monitored by culturing the plant cells on a medium on which the mycobacterium could grow independently, Lowenstein/Jensen medium. It was determined that this mycoplasma is sensitive to TIMENTIN®.

Approximately half the infected cells were washed using polyester support membranes for collection and transfer of cells in the same procedure and medium as described in Example 5. The cells were then distributed into the following treatments:

1. Non-washed:

A) no polyester support; cells placed directly on medium without TIMENTIN® (control)

B) as in A except that TIMENTIN® is incorporated into the gelled medium.

C) as in A except that the cells are placed on a polyester support over the medium.

D) as in B except that the cells are placed on a polyester support over the medium.

E) as in B except that the medium is a biphasic treatment with liquid medium containing TIMENTIN® soaked into a filter paper thin layer under the polyester support. Thiophanate added to the wash medium at 100 mg/L 2. Washed: Treatments F, G, and H were washed as described above prior to plating on the same support and biphasic treatments as C, D, and E respectively. Finally, non-contaminated explants were submitted to all treatments to determine the effects of the treatments on plant regeneration in the absence of mycobacterial contamination. Three plates of each of the four cell lines were placed into each treatment combination.

The explants were then cultured in a dark growth chamber set at 23° C.±2° C. and under low light, and were transferred biweekly using the same support membranes but supplying fresh sterile liquid and gelled media. At each transfer all explants were observed for any mycobacterial regrowth (upon which explants were recorded as contaminated), and a small sample was plated onto the Lowenstein/Jensen indicator medium to detect contamination that may not have been visible on the plant cell media.

The results were that the best decontamination of the pine cell lines was seen in Treatments G and H, in which the washing treatment described in Example 5 was followed by culture of the cells on polyester supports, over either antibiotic-containing medium or biphasic antibiotic-containing medium. Regrowth of the mycobacterium was seen in the majority of the cultures after six weeks in all these treatments, although it was less in the washed treatments F, G and H, indicating that the wash treatment had greatly reduced but not eliminated the inoculum. However, re-application of the antibiotic-containing biphasic medium was completely effective in obviating any further regrowth, causing the wash method combined with the biphasic treatment, both using polyester membrane supports, to be chosen as the most effective improved method for decontamination of these cells.

This example further demonstrates the improved decontamination treatment using biphasic media is suited for use with many types of small explants or callus of any species that can be shown to grow on polyester or fluoropolymer membranes over gelled media, and that the method can be successfully applied to eradication of other types of recurring microbial contaminants from valuable cultures.

### EXAMPLE 11

This example teaches a method for improving the selection of stably transformed cells from among plant tissue cultures. In particular, this example teaches a method of spreading explants such as pieces of petiole and callus generated from such explants, from cottonwood, an angiosperm, onto support membranes for placement over a selection medium, resulting in improved growth of transformed cells. Furthermore, this example teaches an improvement over the traditional method of application of selection agents in the gelled medium, by spreading plant tissue on polyester support membranes laid over a biphasic system incorporating antibiotics in a liquid medium over the gelled medium. This improved method was shown to lead to more effective selection.

This example uses an herbicide as a selection agent. Those skilled in the art of plant transformation recognize that many different selection agents and selection media are appropriate for use with the improved method.

In this example, cottonwood (Populus sp.) cultures were generated as described in Example 1 above by cutting healthy petioles from in vitro shoot cultures onto PHYTAGAR®-gelled DKW medium with DCR vitamins, 0.1 g/L myo-inositol, 3% sucrose, 1 mg/L BAP, and 1 mg/L 2,4-D. These cultures were then transformed with a gene encoding herbicide resistance. Callus that had been selected for herbicide resistance using protocols well known to those skilled in the art of plant transformation was confirmed as transgenic using protocols well known to those skilled in the art of molecular biology. At this time, transgenic callus was used to generate suspension cultures as described in Example 1. Non-transgenic callus from the origin plants was also used to generate suspension cultures as described in Example 1.

Immediately before the start of this experiment, a 1:9 mixture of the transgenic suspension cell line and the corresponding non-transgenic suspension cell line was made as in the preceding Example, in order to simulate, in a controlled fashion, the situation following transformation in which the rare transformed cells must be selected from the abundant non-transformed cells.

The 1:9 mixture was immediately plated on polyester or nylon membrane supports or placed directly on the surface of gelled medium as described in Example 1, with three replicate plates for each treatment and cell line or combination. The medium used was DKW containing a lethal dose of an herbicide (this medium is hereinafter referred to as DKW selection medium), which should kill the non-transformed tissue while allowing growth of the transformed tissue. To verify the ability of the DKW selection medium to kill the non-transformed tissue, non-transformed cells were plated alone on the DKW selection medium using 3 replicate plates per treatment in the same support treatments of nylon, polyester, and directly on the gelled medium. To verify the ability of the DKW selection medium to allow growth of the transformed tissue, transformed cells were plated alone on the DKW selection medium using 3 replicate plates per treatment in the same support treatments of nylon, polyester, and directly on the gelled medium.

The treatments were as follows:

Treatment A: the explants were placed directly on gelled DKW selection medium.

Treatment B: the explants were placed on polyester support membranes placed on gelled DKW selection medium.

Treatment C: the explants were placed on nylon support membranes placed on gelled DKW selection medium.

Treatment D: the polyester support membranes bearing petioles were placed on 1.5 ml liquid DKW medium containing the same concentration of selection agent as the gelled DKW selection medium in the above treatments, pipetted over gelled DKW medium lacking any selection agent.

Treatment E: the polyester support membranes bearing petioles were placed over a filter paper soaked with 1.5 ml liquid DKW medium containing the same concentration of selection agent as the gelled DKW selection medium in the above treatments, laid over gelled DKW medium lacking any selection agent.

Callus cells, or the supports bearing the cells, were placed on petri dishes containing the respective support membrane and biphasic treatments listed above and were incubated in a growth chamber in a 12-hour day, 12-hour night cycle, set at 27±3° C. The callus cells, or the supports bearing the cells, were transferred onto fresh selection medium according to the indicated treatments at intervals of three weeks, and fresh weight of cells for each plate was recorded for a total of nine weeks (Table 18).

TABLE 18
Growth Of Transgenic Callus, Non-Transgenic Callus, And 9:1
Mixtures On Herbicide-Containing Selection Media Over Various
Support Membrane Treatments

| | Average Growth In Grams Over Nine Weeks On | | |
|---|---|---|---|
| | Selection Media Of | | |
| | Non-Transgenic | | |
| | Cells | Transgenic Cells | 9:1 Mixture⁶ |
| Treatment A¹ | 0.07 ± 0.04 | 6.78 ± 2.59 | 3.66 ± 0.64 |
| Treatment B² | 0.82 ± 0.18 | 10.86 ± 3.59 | 7.19 ± 5.21 |
| Treatment C³ | 0.03 ± 0.01 | 8.65 ± 3.73 | 9.31 ± 2.02 |
| Treatment D⁴ | 0.01 ± 0.04 | 8.31 ± 2.07 | 10.66 ± 2.45 |
| Treatment E⁵ | 0.06 ± 0.04 | 11.86 ± 7.02 | 9.06 ± 0.42 |

¹Cells placed directly on gelled suspension media.
²Cells placed on nylon support membrane over gelled selection media.
³Cells placed on polyester support membrane over gelled selection media.
⁴Cells placed on polyester support membrane over biphasic selection media.
⁵Cells placed on polyester support membrane over biphasic selection media in which the liquid phase had been soaked into a filter paper under the support membrane and over the gelled medium.
⁶Mixture of non-transgenic and transgenic cells in a 9:1 ratio.

As can be noted in Table 18, the selection medium was clearly able to stop any substantial growth of non-transgenic cells even when the cells were borne on polyester support membranes over the gelled media or over biphasic selection media. However, non-transgenic cells were able to grow detectably on nylon support membranes over selection medium, indicating that passage of the herbicide was not as efficient through the nylon support membranes as through the polyester support membranes. Thus, when used in a non-simulated selection experiment, the nylon treatment would allow the growth of non-transformed cells within the mixture (which would regenerate “escapes”).

The treatment in which cells were plated directly on the selection medium, the most frequently used method, showed good selection of transformed cells from non-transformed cells. However, the growth of the transgenic material plated directly on the selection medium was inferior to that of the transgenic cells plated on the polyester support membranes. Thus, the polyester support membranes allowed as much effective penetration of the cells by the selection agent as did plating directly on the medium, but allowed better growth and proliferation of the transgenic material selected. As good or even better growth was seen over polyester support membranes placed on biphasic media.

In conclusion, in this example (using simulated selection from a known mixture of herbicide-resistant transgenic cells and herbicide-sensitive non-transgenic cells) we have demonstrated that efficiency of selection of transgenic angiosperm callus was as good when the cells were placed on polyester support membranes, over gelled media, or biphasic media, as when the cells were placed directly on gelled selection media—but the growth was superior when the cells were placed on polyester support membranes over gelled media and as good or even better when the polyester support membranes were placed over biphasic selection media. Furthermore, the efficiency of selection of transgenic angiosperm cells was better with the use of polyester supports than with nylon supports.

The use of the improved selection method described in this experiment has the added benefit that placement of the transgenic cells on non-swelling support membranes, preferably polyester or fluoropolymer support membranes, confers the growth and development advantages seen in Example 3, which are advantageous to the regeneration of transgenic plants.

### EXAMPLE 12

This example teaches the application of a modified selection method, as described in Example 11 above, for improving the selection of stably transformed pine embryogenic tissues. This Example demonstrates that the improved selection method is not limited to angiosperms, to Agrobacterium transformants, or to herbicide selection media, but can be applied to selection of any plant tissue, genetically modified by any method that results in differential sensitivity to a water-soluble selection agent, that can be spread over supports over a selection medium. In particular, this example teaches spreading pine embryogenic tissues on polyester support membranes over selection medium, an improved method that is herein demonstrated to allow less escape of untransformed plant material than unimproved methods in which cells to be subjected to selection are spread directly on selection medium or placed over selection medium on nylon membranes or cellulosic supports such as filter papers.

Stably transformed pine tissue had been generated using a particle bombardment method and selected for the presence of an inserted antibiotic resistance gene, nptII, on DCR₂ gelled medium (Table 5), in this example containing 15 mg/L GENETICIN®, hereinafter referred to as DCR selection medium. Additionally, the presence of an inserted gus gene, encoding a β-glucuronidase enzyme in tissue culture cells, had been detected by deep blue staining of cells from each of the transgenic lines upon exposure to a colorigenic glucuronidase enzyme substrate (commercially available from Inalco, Inc.), according to techniques well known to those skilled in the art of plant transformation, and the presence of both transgenes had been verified by use of the polymerase chain reaction (PCR) using techniques well known to those skilled in the art of molecular biology. The transgenic pine lines containing and expressing both transgenes had then been cryopreserved as described in Example 4 above.

Cells of five transgenic pine lines (lines from four different P. taeda families and one hybrid line), and the corresponding five non-transformed origin lines still held in cryopreservation, were retrieved, proliferated, and placed in suspension as described in Example 4 above. Using the SCV as the basis of calculation, 1:9 mixtures of the transgenic cell line and the corresponding non-transgenic cell line were made, in order to simulate, in a controlled fashion, the situation following transformation in which the rare transformed cells must be selected from the abundant non-transformed cells.

The 1:9 mixtures were immediately plated on polyester, nylon, or filter paper supports or placed directly on the surface of gelled DCR selection medium as described in Examples 4 and 5, with five replicate plates for each treatment and cell line combination. The 15 mg/L GENETICIN® in this medium has been found to be sufficient to kill the non-transformed tissue while allowing growth of the transformed tissue. To verify the ability of the selection medium to kill the non-transformed tissue, non-transformed cells were plated alone on the selection medium using 5 replicate plates per line per treatment in the same support treatments of nylon, polyester, filter paper, and directly on the gelled medium. To verify the ability of the selection medium to allow growth of the transformed tissue, transformed cells were plated alone on the selection medium using 5 replicate plates per line per treatment in the same support treatments of nylon, polyester, filter paper, and directly on the gelled medium.

Cells, or the supports bearing the cells, were transferred onto fresh selection medium at biweekly intervals, and fresh weight of cells for each plate was recorded at each biweekly transfer, for a total of eight weeks (Table 19). Additionally, one plate per line by treatment combination was flooded with X-gluc at 4 weeks, and another at 8 weeks, for examination as to the proportion of cells that stained positive for GUS activity. These data allowed conclusions as to the efficacy of selection of cells submitted to each of the support membrane treatments. As a result of successful selection, growth of non-transgenic cells should be suppressed while growth of transgenic cells should be allowed. Thus it would be expected that plates bearing transgenic cells or a mixture of transgenic and non-transgenic cells would gain weight, while plates bearing only non-transgenic cells should show no weight gain. Furthermore, as a result of successful selection, plates that originally bore a mixture of non-transgenic and transgenic cells should be observed to have only cells staining positively for GUS cells rather than a random mixture of GUS-staining cells with cells that remain colorless upon exposure to X-gluc.

TABLE 19
Growth Of Non-transgenic, Transgenic, And 9:1 Mixed Pine Cell Cultures
On Selection Medium With Various Support Treatments

| | | | | | Cells on nylon | | Cells on polyester | |
|---|---|---|---|---|---|---|---|---|
| | Cells plated directly | | Cells on filter paper | | support membrane | | support membrane | |
| Line or | on medium | | support over medium | | over medium | | over medium | |
| mixture¹ | First 4 wk | All 8 wk | First 4 wk | All 8 wk | First 4 wk | All 8 wk | First 4 wk | All 8 wk |
| P1 | −0.054 ± | −0.075 ± | 0.204 ± | 0.173 ± | 0.006 ± | 0.003 ± | −0.026 ± | −0.020 ± |
| | 0.032 | 0.010 | 0.021 | 0.054 | 0.029 | 0.029 | 0.021 | 0.024 |
| P1-T | 0.088 ± | 4.835 ± | 0.224 ± | 4.458 ± | −0.010 ± | −0.020 ± | 0.062 ± | 3.420 ± |
| | 0.068 | 1.030 | 0.056 | 0.462 | 0.022 | 0.050 | 0.037 | 0.946 |
| 9:1 mixture | −0.054 ± | 0.163 ± | 0.118 ± | 0.225 ± | −0.018 ± | −0.013 ± | 0.012 ± | 0.055 ± |
| P1:P1-T | 0.015 | 0.190 | 0.013 | 0.083 | 0.018 | 0.025 | 0.026 | 0.062 |
| P2 | −0.044 ± | −0.060 ± | 0.130 ± | 0.057 ± | 0.000 ± | 0.005 ± | 0.020 ± | 0.007 ± |
| | 0.018 | 0.008 | 0.016 | 0.064 | 0.024 | 0.031 | 0.010 | 0.013 |
| P2-T | 3.798 ± | 4.538 ± | 3.838 ± | 6.330 ± | 0.150 ± | 3.060 ± | 5.672 ± | 5.553 ± |
| | 0.477 | 0.549 | 1.081 | 1.508 | 0.166 | 1.320 | 0.798 | 0.680 |
| 9:1 mixture | 0.460 ± | 4.840 ± | 0.316 ± | 3.840 ± | −0.002 ± | 0.000 ± | 0.192 ± | 3.650 ± |
| P2:P2-T | 0.399 | 1.523 | 0.130 | 1.008 | 0.031 | 0.024 | 0.068 | 1.603 |
| P3 | −0.047 ± | −0.050 ± | 0.178 ± | 0.123 ± | 0.002 ± | 0.005 ± | 0.008 ± | −0.002 ± |
| | 0.015 | 0.014 | 0.011 | 0.059 | 0.031 | 0.026 | 0.025 | 0.017 |
| P3-T | 0.465 ± | 1.500 ± | 0.612 ± | 2.513 ± | 0.028 ± | 0.120 ± | 0.382 ± | 1.615 ± |
| | 0.093 | 0.367 | 0.248 | 0.406 | 0.058 | 0.174 | 0.138 | 0.369 |
| 9:1 mixture | 0.055 ± | 1.103 ± | 0.274 ± | 1.100 ± | 0.018 ± | 0.050 ± | 0.032 ± | 0.365 ± |
| P3:P3-T | 0.079 | 0.249 | 0.081 | 0.485 | 0.023 | 0.099 | 0.040 | 0.286 |
| P4 | −0.046 ± | −0.060 ± | 0.200 ± | 0.135 ± | −0.046 ± | −0.120 ± | −0.050 ± | −0.073 ± |
| | 0.009 | 0.010 | 0.117 | 0.064 | 0.036 | 0.017 | 0.035 | 0.032 |
| P4-T | 1.662 ± | 2.960 ± | 4.472 ± | 7.160 ± | 0.342± | 3.497 ± | 2.656 ± | 4.563 ± |
| | 0.325 | 0.765 | 2.425 | 1.857 | 0.315 | 1.325 | 1.534 | 0.617 |
| 9:1 mixture | 0.444 ± | 3.207 ± | 1.754 ± | 5.947 ± | −0.006 ± | 3.187 ± | 1.532 ± | 5.530 ± |
| P4:P4-T | 0.165 | 0.206 | 1.554 | 0.821 | 0.067 | 0.451 | 0.990 | 0.718 |
| H1 | −0.034 ± | −0.040 ± | 0.268 ± | 0.210 ± | −0.032 ± | −0.047 ± | 0.092 ± | −0.010 ± |
| | 0.009 | 0.017 | 0.033 | 0.072 | 0.024 | 0.029 | 0.251 | 0.017 |
| H1-T | 2.822 ± | 3.283 ± | 5.830 ± | 4.957 ± | 1.596 ± | 3.947 ± | 3.240 ± | 8.867 ± |
| | 0.388 | 1.167 | 0.900 | 1.364 | 0.935 | 1.540 | 1.940 | 1.835 |
| 9:1 mixture | 0.580 ± | 5.313 ± | 1.160 ± | 9.453 ± | 0.454 ± | 4.670 ± | 1.576 ± | 6.703 ± |
| H1:H1-T | 0.144 | 0.818 | 0.321 | 0.314 | 0.754 | 0.270 | 1.267 | 2.066 |

¹P1 = P. taeda cell line 1, P1-T = P. taeda cell line 1, with inserted nptII and gus, P2 = P. taeda cell line 2, P2-T = P. taeda cell line 2, with inserted nptII and gus, P3 = P. taeda cell line 3, P3-T = P. taeda cell line 3, with inserted nptII and gus, P4 = P. taeda cell line 4, P4-T = P. taeda cell line 4, with inserted nptII and gus, H1 = Hybrid pine
cell line 1, H1-T = Hybrid pine cell line 1, with inserted nptII and gus.

As can be noted in Table 19, and as was also plainly observable in the GUS staining pattern, for all lines as measured at four weeks the filter paper support treatments allowed growth of non-transformed cells as well as transformed cells, clearly failing to “select” only transformed cells. Only following eight weeks of selection could four of the five transgenic lines be distinguished clearly within the mixed cultures on filter paper supports. Growth of all cells was poor on the nylon support membrane treatments, so few transformed cells survived on the nylon support membranes. Only one transgenic line was growing detectably on the nylon support membranes at four weeks, while even at eight weeks only three of the five lines had been able to grow on the nylon membranes. As with the filter paper treatments, in the nylon support treatments the GUS staining pattern demonstrated that the growing cultures contained non-transformed, non-staining cells. Thus, the nylon support membrane treatments also resulted in capture of only 60% of the actual number of transformed cell lines from the mixtures, and the “captured” events remained chimaeric. In conclusion, both the nylon and filter paper treatments, often used for selection of plant tissue cultures, required lengthier exposure to the selection medium in order to capture the majority of transformation events, allowed the growth of non-transformed cells within the mixture (which would regenerate “escapes”), and were unable to capture all transformation events.

Over the polyester support membranes, in contrast, three of the five transformed lines were distinguishable by growth at the end of four weeks, and the GUS staining patterns revealed that the growing cells were expressing GUS uniformly. At the end of eight weeks, all five of the transformant lines had been successfully selected from their non-transformed counterparts.

The treatment in which cells were plated directly on the selection medium, another often used method, showed good selection of transformed cells from non-transformed cells. At four weeks into selection, three of the five transgenic cell lines could be distinguished by growth exceeding that of non-transgenic cell lines, and at eight weeks into selection, all five transgenic lines could be distinguished. However, only in one of the five lines was the growth of the transgenic material plated directly on the selection medium comparable to that plated on the polyester support membranes. In conclusion, the polyester support membranes allowed as much effective penetration of the cells by the selection agent as did plating directly on the medium, but allowed better growth and proliferation of the transgenic material selected.

The results were that transformed cells, as evidenced by histochemical staining for the expression of a reporter gene, were able to grow to greater weights over polyester membranes than over nylon membranes, filter paper, or over gelled media without fabric supports. Furthermore, untransformed cells were not seen in as great a frequency over polyester membranes as over nylon membranes or over filter paper supports. This indicates that the efficiency of selection was improved with the use of polyester supports.

The improved selection method described in this example has been extended to selection of transformants in a variety of species.

### EXAMPLE 13

This Example further demonstrates that the application of the improved selection method described in Examples 11-12 above enables the improved selection of other species following transformation by other means.

Cells of four transgenic P. radiata lines and the corresponding non-transformed origin lines were placed in suspension as described in Example 12 above, using the SCV as the basis of calculation, in 1:9 mixtures of the transgenic cell line and the corresponding non-transgenic cell line, in order to simulate, in a controlled fashion, the situation following transformation in which the rare transformed cells must be selected from the abundant non-transformed cells.

The 1:9 mixtures were immediately plated on polyester, nylon, or filter paper supports or placed directly on the surface of gelled medium as described in Example 11, with five replicate plates for each treatment and cell line combination. The medium used was P. radiata maintenance medium as described in Example 5, containing 15 mg/L GENETICIN®, hereinafter referred to as P. radiata selection medium, which should kill the non-transformed tissue while allowing growth of the transformed tissue. Those skilled in the art of plant transformation will recognize that many different selection media formulations may be suitable for use in the present method. To verify the ability of the P. radiata selection medium to kill the non-transformed tissue, non-transformed cells were plated alone on the P. radiata selection medium using 5 replicate plates per line per treatment in the same support treatments of nylon, polyester, filter paper, and directly on the gelled medium. To verify the ability of the P. radiata selection medium to allow growth of the transformed tissue, transformed cells were plated alone on the P. radiata selection medium using 5 replicate plates per line per treatment in the same support treatments of nylon, polyester, filter paper, and directly on the gelled medium.

As in Example 11, cells or the supports bearing the cells, were transferred onto fresh P. radiata selection medium at biweekly intervals, and fresh weight of cells for each plate was recorded at each biweekly transfer, for a total of eight weeks. Additionally, one plate per line by treatment combination was flooded with X-gluc at 4 weeks, and another at 8 weeks, for examination as to the proportion of cells that stained positive for GUS activity. These data allowed conclusions as to the efficacy of selection of cells submitted to each of the support membrane treatments. As a result of successful selection, growth of non-transgenic cells should be suppressed while growth of transgenic cells should be allowed. Thus it would be expected that plates bearing transgenic cells or a mixture of transgenic and non-transgenic cells would gain weight, while plates bearing only non-transgenic cells should show no weight gain. Furthermore, as a result of successful selection, plates that originally bore a mixture of non-transgenic and transgenic cells should be observed to have only cells staining positively for GUS cells rather than a random mixture of GUS-staining cells with cells that remain colorless upon exposure to X-gluc.

As was noted in Example 12, for all lines as measured at four weeks the filter paper support treatments allowed growth of non-transformed cells as well as transformed cells, clearly failing to “select” only transformed cells. Only following eight weeks of selection could the transgenic lines be distinguished clearly within the four mixed cultures on filter paper supports. Accordingly, it could be concluded that the filter paper treatments, often used for selection of plant tissue cultures, required lengthier exposure to the selection medium in order to capture the majority of transformation events and allowed the growth of non-transformed cells within the mixture (which would regenerate “escapes”).

Over the polyester support membranes, in contrast, all four transformed lines were distinguishable by growth at the end of four weeks. Total growth of the P. radiata cells was also best on the polyester support membranes, resulting in both the best selection and the best growth of transformed pine cells being seen in this treatment. This indicates that the efficiency of selection was significantly improved with the use of polyester supports.

Over the polyester support membranes, in contrast, all four transformed lines were distinguishable by growth at the end of four weeks. Total growth of the P. radiata cells was also best on the polyester support membranes, whether over biphasic media incorporating a filter paper underlay or not, resulting in both the best selection and the best growth of transformed pine cells being seen in this treatment. This indicates that the efficiency of selection was significantly improved with the use of polyester supports.

### EXAMPLE 14

This example teaches a method for improving the administration of various components of a plant tissue culture medium, which may be large or heat-sensitive molecules such as plant growth regulators or polymers, by incorporating these molecules, normally administered in a gelled medium, into a liquid medium pipetted over a gelled medium lacking these molecules, forming a biphasic system on which polyester support membranes bearing plant tissue may be laid, facilitating efficient diffusion of the large molecules into the plant material.

Loblolly pine embryogenic cell cultures were initiated, proliferated, cryopreserved, retrieved, further proliferated on gelled maintenance medium, and placed into liquid suspension for plating on embryo development medium MSG₁ as described in Examples 3-4 above. Three replicate plates of each line were prepared for each of the following treatments:

Treatment A: the cells were plated onto polyester support membranes laid onto the surface of gelled embryo development medium MSG₁.

Treatment B: the cells were plated onto polyester support membranes laid onto 1 ml liquid embryo development medium MSG₁ containing PEG but lacking gelling agent and activated charcoal, pipetted over gelled embryo development medium MSG₁

Treatment C: the cells were plated onto polyester support membranes laid onto 1 ml liquid embryo development medium MSG₁ containing PEG but lacking gelling agent and activated charcoal, pipetted over gelled embryo development medium MSG₁ lacking abscisic acid.

Abscisic acid (ABA) is important in embryo development, as described in U.S. Pat. Nos. 5,036,007 (Gupta and Pullman 1991), 5,236,841 (Gupta and Pullman 1993), and 5,856,191 (Handley 1999). Continued high concentrations of ABA are optimal for terminal embryo development in Southern yellow pines such as P. taeda, as described in U.S. Pat. No. 5,856,191 (Handley 1999).

The purpose of the latter treatment was to determine whether abscisic acid, an expensive and possibly partially labile component, could be administered via a thin layer in a biphasic medium, thus allowing use of a smaller volume of medium containing an accordingly smaller amount of ABA while still maintaining a high and continuous concentration of ABA administered to the developing embryos.

Embryos were developed, harvested and germinated as described in Example 4 above. The results of the counts of high quality cotyledonary stage embryos, and the germination tests with these embryos, are shown in Table 20 below.

TABLE 20
Effects Of Abscisic Acid Applied In A Liquid Phase Under
Polyester Support Membranes And Over Gelled Development
And Maturation Media

| | Average Number Of High Quality Cotyledonary Embryos Per | | |
|---|---|---|---|
| | Plate Developed Over Different Biphasic Media Treatments | | |
| | under Polyester Membranes (Percentage Of Resulting Embryos | | |
| P. taeda | That Germinated) | | |
| Cell | | | Gelled And |
| Line | Gelled Phase¹ | Liquid Phase² | Liquid Phase³ |
| P1 | 91.7 ± 28.4 (42.5%) | 44.3 ± 21.1 (55.0%) | 30.3 ± 7.1 (52.5%) |
| P2 | 68.0 ± 1.7 (62.5%) | 44.3 ± 4.9 (67.5%) | 48.7 ± 5.0 (57.5%) |

¹Gelled phase only (no liquid phase).
²Liquid phase with same abscisic acid content as gelled phase.
³Gelled phase lacking abscisic acid and liquid phase containing abscisic acid.

The results were that the culture of cells over a bi-layer formed by liquid embryo development medium MSG₁ containing PEG, pipetted in a thin film over gelled embryo development medium MSG₁ and under polyester support membranes, allowed the development of harvestable embryos of normal appearance. While the embryos per plate were decreased by 30-60%, normal development was accomplished with the overall use of 5% of the amount of abscisic acid. If the cost of ABA is high or the supply is limited, this method could still be of utility in the production of regenerated plants. Furthermore, the ABA used in the liquid phase was not subjected to the high gel polymerization temperature encountered when it is incorporated into the gel phase.

Furthermore, in neither of these two loblolly pine lines were the percentages of embryos germinating following these treatments, even using a greatly reduced amount (5%) of ABA, much reduced when compared to the percentage germination found in the control treatment lacking a liquid phase.

These results demonstrate that the incorporation of a plant growth regulator or other large molecule into a liquid phase under a polyester support membrane can constitute a useful method for administering said molecule to cells without damage to the molecule or significant detriment to its action on the cells.

In another experiment, an attempt had been made to decrease the osmotic potential of the liquid phase in order to facilitate filter sterilization. The results are shown in Table 21.

TABLE 21
Effects of Different Biphasic Media Under Polyester Membranes

| | Average Number Of High Quality Cotyledonary Embryos | | |
|---|---|---|---|
| | Per Plate Developed Over Different Biphasic Media Under | | |
| | Polyester Membranes | | |
| | | Liquid Phase | Liquid Phase |
| P. taeda | | containing ABA | containing ABA |
| Cell Line | Gelled Phase | but no PEG | and PEG |
| L42 | 14.7 ± 10.8 | 0 | 0 |
| L9 | 83.7 ± 8.6 | 97.0 ± 20.7 | 64.0 ± 23.6 |
| L5 | 336.7 ± 51.3 | 201.0 ± 44.5 | 26.3 ± 45.6 |

As seen in this table, the application of a liquid medium in a biphasic system was more successful when PEG was a component of the liquid medium. However, it was desirable to decrease the osmotic potential of the liquid phase in order to facilitate filter sterilization of the liquid medium. In a second experiment, this was done by decreasing or eliminating the sugar in the liquid phase, and continuous supply of the liquid phase of the above biphasic treatments was enabled by soaking the liquid phase into a filter paper placed between the gelled medium phase and the polyester support membrane. Accordingly, the three treatments listed in Table 20 above were repeated and additional treatments were added, comprising the following:

Treatment D: the cells were plated onto polyester support membranes laid onto a filter paper soaked with 1 ml liquid embryo development medium MSG₁ containing PEG but lacking sugar (maltose), gelling agent and activated charcoal, pipetted over gelled embryo development medium MSG₁ lacking abscisic acid.

Treatment E: the cells were plated onto polyester support membranes laid onto a filter paper soaked with 1 ml liquid embryo development medium MSG₁ containing PEG but having only 2% sugar (maltose), gelling agent and activated charcoal, pipetted over gelled embryo development medium MSG₁ lacking abscisic acid.

The results were that each of the two treatments D and E were similarly able to support development of somatic embryos, demonstrating that the improved polyester support membrane method for administration of plant growth regulators facilitates administration of the ABA in a separate medium that does not contain maltose, and this ABA, contained in the separate lower volume medium, is sufficient to enable terminal embryo development. Embryo development does not require incorporation of any ABA into the gelled medium containing the other nutrients and maltose.

Accordingly, this improved method for the administration of plant growth regulators, using a biphasic system in which the tissue is borne on a polyester support membrane over ap biphasic system in which two separate media are used, one of which contains maltose but lacks ABA, and the other of which contains ABA but lacks maltose, both containing PEG, has been shown to be enabling for the development of mature harvestable cotyledonary embryos for the production of planting stock.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

What is claimed is:

1. An improved method for plant cell tissue culture which comprises culturing plant cells on a support membrane placed over a gel medium, wherein the improvement comprises the use of a liquid-permeable support membrane prepared from low-absorption fibers.

2. The method of claim 1, wherein said support membrane is prepared from material selected from the group consisting of polyesters, polypropylenes, fluoropolymers, and combinations thereof.

3. The method of claim 1, wherein said support membrane is placed over a layer containing one or more culture components and said layer is positioned on said gel medium.

4. The method of claim 1, wherein said plant cells are plated onto said support membrane prior to culturing.

5. The method of claim 3, wherein said layer is a thin film of liquid medium.

6. The method of claim 3, wherein said layer is a filter paper with a liquid medium absorbed therein.

7. A plant cell tissue culture prepared by the method of claim 1.

8. An improved method for growing plant cell tissues which comprises culturing plant cells on a support membrane placed over a gel medium, wherein the improvement comprises the use of a liquid-permeable support membrane prepared from low-absorption fibers that permits the diffusion of components from the medium to the plant cells to promote the growth of the plant cells above the medium without allowing the plant cells to become embedded in the medium.

9. The method of claim 8, wherein said support membrane is prepared from material selected from the group consisting of polyesters, polypropylenes, fluoropolymers, and combinations thereof.

10. The method of claim 8, wherein said support membrane is placed over a layer containing one or more culture components and said layer is positioned on said gel medium.

11. The method of claim 8, wherein said plant cells are plated onto said support membrane prior to culturing.

12. The method of claim 10, wherein said layer is a thin film of liquid medium.

13. The method of claim 10, wherein said layer is a filter paper with a liquid medium absorbed therein.

14. The method of claim 10, wherein said layer contains one or more plant growth regulators which are not also contained in said solid gel medium.

15. The method of claim 14, wherein the plant growth regulator is absicic acid.

16. A plant cell tissue prepared by the method of claim 8.

17. An improved method for regenerating plants which comprises:
(a) culturing plant cells on a support membrane placed over a gel medium to produce embryos or shoots, wherein the improvement comprises the use of a liquid-permeable support membrane prepared from low-absorption fibers that permits the diffusion of components from the medium to the plant cells to promote the growth of the plant cells above the medium without allowing the plant cells to become embedded in the medium, and
(b) germinating said embryos or rooting said shoots to produce plants.

18. The method of claim 17, wherein said support membrane is prepared from material selected from the group consisting of polyesters, polypropylenes, fluoropolymers, and combinations thereof.

19. The method of claim 17, wherein said support membrane is placed over a layer containing one or more culture components and said layer is positioned on said gel medium.

20. The method of claim 17, wherein said plant cells are plated onto said support membrane prior to culturing.

21. The method of claim 19, wherein said layer is a thin film of liquid medium.

22. The method of claim 19, wherein said layer is a filter paper with a liquid medium absorbed therein.

23. A plant prepared by the method of claim 17.

24. An improved method for selecting genetically transformed plant cells which comprises:
(a) culturing plant cells which have been subjected to transformation on a support membrane placed over a gel medium, wherein the improvement comprises the use of a liquid-permeable support membrane prepared from material selected from the group consisting of polyesters, polypropylenes, fluoropolymers, and combinations thereof;
(b) contacting said plant cells with a selection agent; and
(c) selecting transformed plant cells.

25. The method of claim 24, wherein said selection agent is contained in said gel medium.

26. The method of claim 24, wherein said selection agent is contained in a layer and said support membrane is placed over said layer which is positioned on said gel medium

27. The method of claim 26, wherein said layer is a thin film of liquid medium.

28. The method of claim 26, wherein said layer is a filter paper with a liquid medium absorbed therein.

29. A transformed plant cell prepared by the method of claim 24.

30. An improved method for eradicating a bacterial contaminant or a fungal contaminant from plant cells which comprises:
(a) culturing plant cells on a liquid-permeable support membrane over a layer containing an eradicant, wherein the improvement comprises the use of a liquid-permeable support membrane prepared from material selected from the group consisting of polyesters, polypropylenes, fluoropolymers, and combinations thereof, and said layer is positioned over a gel medium; and
(b) recovering plant cells from which said bacterial contaminant or fungal contaminant has been eradicated.

31. The method of claim 30, wherein said layer is a thin film of liquid medium.

32. The method of claim 30, wherein said layer is a filter paper with a liquid medium absorbed therein.

33. A plant cell prepared by the method of claim 30.

34. A method for eradicating a bacterial contaminant or a fungal contaminant from plant cells which comprises:
(a) washing contaminated plant cells in a liquid wash medium;
(b) plating said cells on a liquid-permeable support membrane prepared from material selected from the group consisting of polyesters, polypropylenes, fluoropolymers, and combinations thereof;
(c) recovering decontaminated plant cells with minimal physical damage.

35. The method of claim 34, wherein plant cells are plated onto said support membrane prior to step (a).

36. The method of claim 34, wherein the liquid wash medium contains one or more eradicants selected from the group consisting of antibiotics, bacteriostatic agents, antifungal agents, fungistatic agents, and combinations thereof.

37. The method of claim 34, wherein each wash is carried out for a duration sufficient to expose all the plant cells to the wash medium without interfering with subsequent growth of the plant cells.

38. The method of claim 34, wherein steps (a) and (b) are repeated until microbial contamination is no longer detectable.

39. A plant cell prepared by the method of claim 34.

40. A method for eradicating a bacterial contaminant or a fungal contaminant from plant cells which comprises:
(a) washing contaminated plant cells in a liquid wash medium for a duration sufficient to expose all the plant cells to the wash medium without interfering with subsequent growth of the plant cells;
(b) plating said plant cells on a liquid-permeable support membrane prepared from material selected from the group consisting of polyesters, polypropylenes, fluoropolymers, and combinations thereof;
(c) culturing said plant cells on said support membrane over a layer containing an eradicant, wherein said layer is positioned over a gel medium; and
(d) recovering plant cells from which said bacterial contaminant or fungal contaminant has been eradicated.

41. The method of claim 40, wherein steps (a) and (b) are repeated until microbial contamination is obviously considerably decreased.

42. The method of claim 40, wherein said layer is a thin film of liquid medium.

43. The method of claim 40, wherein said layer is a filter paper with a liquid medium absorbed therein.

44. A plant cell prepared by the method of claim 40.

45. An improved method for producing transgenic plants which comprises:
(a) culturing plant cells which have been subjected to transformation on a liquid-permeable support membrane placed over a first gel medium, wherein the first improvement comprises the use of a liquid-permeable support membrane prepared from material selected from the group consisting of polyesters, polypropylenes, fluoropolymer fabrics, and combinations thereof;
(b) contacting said plant cells with a selection agent;
(c) selecting transformed plant cells;
(d) promoting the production of embryos or shoots from said transformed plant cells by culturing the transformed plant cells on a second support membrane placed over a gel medium, wherein the second improvement comprises the use of a liquid-permeable second support membrane prepared from low-absorption fibers that permits the diffusion of components from the medium to the plant cells to promote the growth of the plant cells above the medium without allowing the plant cells to become embedded in the medium, and
(e) rooting said shoots or germinating said embryos to produce transgenic plants.

46. The method of claim 45, wherein the second liquid-permeable support membrane is prepared from material selected from the group consisting of polyesters, polypropylenes, fluoropolymers, and combinations thereof.

47. The method of claim 45, wherein said selection agent is contained in said first gel medium.

48. The method of claim 45, wherein said selection agent is contained in a layer and said support membrane is placed over said layer which is positioned on said first gel medium

49. The method of claim 48, wherein said layer is a thin film of liquid medium.

50. The method of claim 48, wherein said layer is a filter paper with a liquid medium absorbed therein.

51. The method of claim 45, wherein said second support membrane is placed over a layer containing one or more culture components and said layer is positioned on said second gel medium.

52. The method of claim 45, wherein said transformed plant cells are plated onto said second support membrane prior to culturing.

53. The method of claim 51, wherein said layer is a thin film of liquid medium.

54. The method of claim 51, wherein said layer is a filter paper with a liquid medium absorbed therein.

55. A transgenic plant prepared by the method of claim 45.
